SEQUENCE LISTING (30 sequences)

SEQ ID NO: 1 - DNA, 25 bp, Artificial Sequence
  Genotyping primer 1
aacttcactgctacatctccatggc

SEQ ID NO: 2 - DNA, 25 bp, Artificial Sequence
  Genotyping primer 2
gctcaaggttgtatgccttggtgct

SEQ ID NO: 3 - DNA, 26 bp, Artificial Sequence
  Genotyping primer 3
tgcatcgcattgtctgagtaggtgtc

SEQ ID NO: 4 - DNA, 20 bp, Artificial Sequence
  GAPDH Forward primer
ttgaggtcaatgaaggggtc

SEQ ID NO: 5 - DNA, 20 bp, Artificial Sequence
  GAPDH Reverse primer
gaaggtgaaggtcggagtca

SEQ ID NO: 6 - DNA, 19 bp, Artificial Sequence
  MAP2 Forward primer
caacggagagctgacctca

SEQ ID NO: 7 - DNA, 21 bp, Artificial Sequence
  MAP2 Reverse primer
ctacagcctcagcagtgacta

SEQ ID NO: 8 - DNA, 20 bp, Artificial Sequence
  BKa Forward primer
acaagtctgccaaccgagag

SEQ ID NO: 9 - DNA, 20 bp, Artificial Sequence
  BKa Reverse primer
atggacatcttgggccgttt

SEQ ID NO: 10 - DNA, 21 bp, Artificial Sequence
  ATPase a1 Forward primer
tgctctgtgcttttctctctg

SEQ ID NO: 11 - DNA, 21 bp, Artificial Sequence
  ATPase a1 Reverse primer
ttcagttcatccatgtccctg

SEQ ID NO: 12 - DNA, 20 bp, Artificial Sequence
  ATPase a2 Reverse primer
gcgtgtgctgggattctgtc

SEQ ID NO: 13 - DNA, 20 bp, Artificial Sequence
  ATPase a2 Reverse primer
tggccttggctgtgataggg

SEQ ID NO: 14 - DNA, 21 bp, Artificial Sequence
  ATPase b1 Forward primer
aacctaagcctcccaagaatg

SEQ ID NO: 15 - DNA, 21 bp, Artificial Sequence
  ATPase b1 Reverse primer
caggagtttgccatagtacgg

SEQ ID NO: 16 - DNA, 20 bp, Artificial Sequence
  ATPase b2 Forward primer
ttcgtgtggaacccgaggac

SEQ ID NO: 17 - DNA, 20 bp, Artificial Sequence
  ATPase b2 Reverse primer
gcgaatcatcaagcccggtg

SEQ ID NO: 18 - DNA, 33 bp, Artificial Sequence
  Adaptor 1
agatcggaagagcacacgtctgaactccagtca

SEQ ID NO: 19 - DNA, 33 bp, Artificial Sequence
  Adaptor 2
agatcggaagagcgtcgtgtagggaaagagtgt

SEQ ID NO: 20 - DNA, 23 bp, Artificial Sequence
  sgRNA sequence
ctactaccaccagttaactgagg

SEQ ID NO: 21 - DNA, 23 bp, Artificial Sequence
  Off-target 1
ctgagaccactagttaactgtgg

SEQ ID NO: 22 - DNA, 23 bp, Artificial Sequence
  Off-target 2
ctcctaaaactagttaactgtag

SEQ ID NO: 23 - DNA, 23 bp, Artificial Sequence
  Off-target 3
caaataccagcagttcactggag

SEQ ID NO: 24 - DNA, 23 bp, Artificial Sequence
  Off-target 4
ctagtcacaccagttacctgaag

SEQ ID NO: 25 - DNA, 23 bp, Artificial Sequence
  Off-target 5
ctccaacccccagtgaactgtgg

SEQ ID NO: 26 - DNA, 23 bp, Artificial Sequence
  Off-target 6
ctacaaccaccagtttactgcag

SEQ ID NO: 27 - DNA, 23 bp, Artificial Sequence
  Off-target 7
ctgcaaccactagttaactggag

SEQ ID NO: 28 - DNA, 23 bp, Artificial Sequence
  Off-target 8
cttctaccctcagttaactgcag

SEQ ID NO: 29 - DNA, 23 bp, Artificial Sequence
  Off-target 9
tttcaacaaccagttaactggag

SEQ ID NO: 30 - DNA, 23 bp, Artificial Sequence
  Off-target 10
ctattaccagcagttatctgagg

(19) United States
(12) Patent Application Publication
(10) Pub. No.: US 2023/0042176 A1
(43) Pub. Date: Feb. 9, 2023

(54) METHOD FOR TREATING ANGELMAN SYNDROME AND RELATED DISORDERS

(71) Applicants: National University of Singapore, Singapore (SG); Singapore Health Services PTE Ltd, Singapore (SG); Agency for Science, Technology and Research, Singapore (SG)

(72) Inventors: Hyunsoo Shawn JE, Singapore (SG); Qiang YUAN, Singapore (SG); Xuyang SUN, Singapore (SG)

(21) Appl. No.: 17/786,384

(22) PCT Filed: Dec. 18, 2020
(86) PCT No.: PCT/SG2020/050762

§ 371 Date: Jun. 16, 2022

Related U.S. Application Data
(60) Provisional application No. 62/949,603, filed on Dec. 18, 2019.

(51) Int. Cl. A61K 38/17; A61K 31/407; A61K 31/53; C12N 15/113; A61P 25/08

(52) U.S. Cl. CPC A61K 38/1767; A61K 31/407; A61K 31/53; C12N 15/1138; A61P 25/08; C12N 2310/14; C12N 2310/16; C12N 2310/11; C12N 2310/12

(57) ABSTRACT

The present invention relates to methods of treating or ameliorating seizures relating to disruptions in Ubiquitin Protein Ligase E3A (UBE3A) gene. More particularly, the invention relates to the use of BK channel antagonists for the prophylaxis or treatment of seizures in a subject with Angelman syndrome or related autism spectrum disorder (ASD). In some embodiments, BK channel antagonist is Paxilline, iberiotoxin (IBTX) or GAL-021.

### FIELD OF THE INVENTION

The present invention relates to methods of treating or ameliorating seizures relating to disruptions in Ubiquitin Protein Ligase E3A (UBE3A) gene. More particularly, the invention relates to the use of BK channel antagonists for the prophylaxis or treatment of seizures in a subject with Angelman syndrome.

### BACKGROUND OF THE INVENTION

Angelman syndrome (AS) is an autism spectrum disorder (ASD) characterized by delayed development, intellectual disability, and frequent episodes of seizures (Buiting et al., Nat Rev Neurol 12, 584-593 (2016)). Approximately 90% of AS cases are caused by the loss of function of the UBE3A gene, which encodes an HECT E3 ubiquitin ligase (Mabb et al., Trends Neurosci 34, 293-303 (2011)). It has been postulated that the loss of the UBE3A protein could result in the build-up of AS-relevant substrate proteins and thereby contribute to disease pathogenesis (Sell and Margolis, Front Neurosci 9, 322 (2015)). Previous studies that have used multiple lines of AS model mice have provided a number of mechanistic insights by demonstrating impaired synaptic connectivity, an imbalance between network excitation and inhibition, and delayed neurodevelopmental processes (Jiang et al., 1998a; Judson et al., J Neurosci 37, 7347-7361 (2017); Judson et al., Neuron 90, 56-69 (2016); Wallace et al., Neuron 74, 793-800 (2012); Wallace et al., J Neurophysiol, 118(1): 634-646 (2017)). However, no concurrent mechanism has been fully established to underlie epilepsy, a common feature in AS patients. Likewise, although recent advances achieved in studies using AS patient-derived induced pluripotent stem cells (AS-iPSCs) and differentiated neurons have identified several cellular deficits, neither the pathological mechanism underlying AS nor the biological substrate(s) of UBE3A have been characterized (Chamberlain et al., Proc Natl Acad Sci USA 107, 17668-17673 (2010); Fink et al., Nat Commun 8, 15038 (2017)).

Genetic engineering approaches have been used to rescue the neurological deficits in AS model mice (Ube3a-deficient mice), but this approach relies on knocking out genes, making it impractical in humans. A pharmacological approach offers a more viable alternative. A pharmacological approach to activate imprinted genes (e.g., the paternal allele of UBE3A silenced through epigenetic imprinting) for AS is available, but the drug (topoisomerase inhibitors) targets and affects the transcription of many genes other than UBE3A, thereby producing unwanted side effects (e.g. cancer).

Previous studies performed in AS mouse models (Ube3a-deficient mice) have shown that these mice exhibit various phenotypic differences ranging from abnormal dendritic arborization to impaired synaptic plasticity. However, these reports have not extensively characterized a concurrent mechanism to explain how the network hyperactivity observed in AS arises as a result of UBE3A deficiency in individual neurons.

There is a need to develop alternative or improved methods to ameliorate the negative effects loss of UBE3A protein has on cells carrying UBE3A mutations. The present disclosure aims at providing such a method.

### SUMMARY OF THE INVENTION

Disruption of the UBE3A gene leads to accumulation of big potassium channels (BK channels), which increases the likelihood of seizures, particularly in subjects with Angelman Syndrome. Surprisingly, the inventors have found that the use of inhibitors of BK channel activity can reduce the network hyperactivity and seizures in said subjects. Without being bound by theory, the inventors submit that the whole basis of the therapy is that disrupting BK channel function suppresses the physiological effects of UBE3A mutation and reduces seizures.

Accordingly, in a first aspect the present invention provides a compound or composition comprising said compound for use in the prophylaxis or treatment of seizures caused by one or more UBE3A mutations in a subject.

In some embodiments, the one or more UBE3A mutations cause a BK channelopathy.

In some embodiments, the compound or composition is an antagonist of BK channel activity.

In some embodiments, the subject has Angelman syndrome or a related autism spectrum disorder.

References herein (in any aspect or embodiment of the invention) to antagonist compounds of BK channels includes references to such compounds per se, to tautomers of such compounds, as well as to pharmaceutically acceptable salts or solvates, or pharmaceutically functional derivatives of such compounds.

In some embodiments the compound is selected from the group comprising Paxilline, IBTX and GAL-021.

Paxilline has the IUPAC name (2R,4bS,6aS,12bS,12cR,14aS)-4b-hydroxy-2-(1-hydroxy-1-methylethyl)-12b,12c-dimethyl-5,6,6a,7,12,12b,12c,13,14,14a-decahydro-2H-chromeno[5′,6′:6,7]indeno[1,2-b]indol-3(4bH)-one.

Paxilline has the structure:

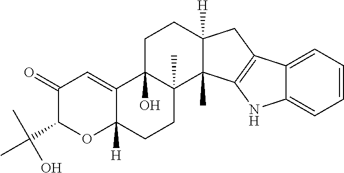

IBTX (synonym Iberiotoxin) is a peptide and has the IUPAC condensed name H-Pyr-D-Phe-D-Thr-D-Asp-D-Val-Asp-D-Cys(1)-Ser-Val-Ser-Lys-Glu-Cys(2)-D-Trp-D-Ser-Val-D-Cys(3)-Lys-Asp-Leu-Phe-Gly-Val-Asp-Arg-Gly-Lys-Cys(1)-Met-Gly-Lys-Lys-D-Cys(2)-D-Arg-D-Cys(3)-D-Tyr-D-Gln-OH.

GAL-021 has the IUPAC name 2-N-methoxy-2-N-methyl-4-N,6-N-dipropyl-1,3,5-triazine-2,4,6-triamine; and the structure;

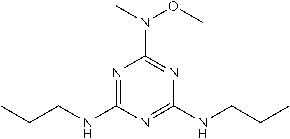 [SMILES: CCCNc1nc(NCCC)nc(N(C)OC)n1]

Alternative compounds of the present invention include small molecules and functional nucleic acids. Functional nucleic acids are nucleic acid molecules that carry out a specific function in a cell, such as binding a target molecule or catalyzing a specific reaction.

Such functional nucleic acids may inhibit the activity of the BK channel. Functional nucleic acids include but are not limited to antisense molecules, aptamers, ribozymes, triplex forming molecules, small interfering RNA (siRNA), other forms of RNA interference (RNAi), and external guide sequences (EGS). In one embodiment, a siRNA could be used to reduce or eliminate expression of the target molecule.

Aptamers are molecules that interact with a target nucleic acid, preferably in a specific way. Typically, aptamers are small nucleic acids ranging from 15-50 bases in length that fold into defined secondary and tertiary structures, such as stem-loops or G-quartets. Representative examples of how to make and use aptamers to bind a variety of different target molecules can be found in, for example, U.S. Pat. Nos. 5,476,766 and 6,051,698 (which are hereby incorporated by reference only for this teaching). The secondary structure may inhibit expression of a polypeptide encoded by a gene or inhibit the function of a polypeptide itself. Aptamers bind to these specific targets because of electrostatic interactions, hydrophobic interactions, and their complementary shapes. Aptamers of the present disclosure may interact with a nucleic acid encoding a target molecule, such as a BK channel polypeptide component, or may block the function of the BK channel.

In some embodiments, the composition of any aspect of the invention comprises pharmaceutically acceptable salts or solvates, or pharmaceutically functional derivatives of said BK antagonist compound.

In some embodiments, said composition comprises a BK channel antagonist compound with a pharmaceutically-acceptable adjuvant, diluent or carrier.

In some embodiments, the composition is formulated for administration of a BK channel antagonist in the range of about 0.05 mg/kg to about 10 mg/kg, about 0.05 mg/kg to about 5 mg/kg, preferably about 0.3 mg/kg to about 3 mg/kg.

McLeod et al., (British J. Anaesthesia 113, 875-883 (2014)) has shown that healthy human volunteers can tolerate GAL-021 i.v. infused at a dosage of 0.1-0.96 mg/kg/hr for 1 hour and intermediate doses up to 4 h.

Another aspect of the invention provides a method of prophylaxis or treatment of seizures in a subject, comprising administering to a subject in need thereof a therapeutically effective amount of an antagonist of BK channel activity.

In some embodiments, one or more UBE3A mutations cause a BK channelopathy in the subject.

In some embodiments, the subject has Angelman syndrome or a related autism spectrum disorder.

In some embodiments, the antagonist of BK channel activity is selected from the group comprising Paxilline, IBTX, GAL-021, small molecules, and functional nucleic acids such as antisense molecules, aptamers, ribozymes, triplex forming molecules, small interfering RNA (siRNA), other forms of RNA interference (RNAi), and external guide sequences (EGS).

In some embodiments, the therapeutically effective amount is in the range of 0.05 mg/kg to about 10 mg/kg, about 0.05 mg/kg to about 5 mg/kg, preferably about 0.3 mg/kg to about 3 mg/kg.

Another aspect of the invention provides use of a BK channel antagonist compound or composition of any aspect of the invention for the manufacture of a medicament for the prophylaxis or treatment of seizures in a subject.

In some embodiments the seizures are caused by one or more UBE3A mutations.

In some embodiments the subject has Angelman syndrome or a related autism spectrum disorder.

In some embodiments, the antagonist of BK channel activity is selected from the group comprising Paxilline, IBTX, GAL-021, small molecules, and functional nucleic acids such as antisense molecules, aptamers, ribozymes, triplex forming molecules, small interfering RNA (siRNA), other forms of RNA interference (RNAi), and external guide sequences (EGS).

### BRIEF DESCRIPTION OF THE FIGURES

FIGS. 1A-1N show altered functional properties of UBE3A-deficient human neurons. (FIGS. 1A-1D) Generation of human induced neurons with UBE3A knockout (KO) human embryonic stem cells (hESCs). (FIG. 1A) Schematic illustrating the CRISPR/Cas9-mediated gene editing approach used to knock out UBE3A in hESCs (top) and immunoblot showing the absence of UBE3A protein in UBE3A KO hESCs (bottom). (FIG. 1B) Schematic illustrating the protocol used to generate the human neurons used in this study (top) and representative morphological images of day 35 WT and KO neurons immunostained for MAP2 (axon) and Synapsin (synaptic boutons) (bottom). Scale bar: 20 μm. (FIG. 1C) Sholl analysis of WT and KO induced neurons at day 35. (FIG. 1D) Representative images and quantification of synaptic puncta density calculated from neurons immunostained for Synapsin. Scale bar: 1 μm. (FIGS. 1E-1I) Altered excitability in KO neurons (H9-derived). (FIG. 1E) F-I curves showing spike frequency versus current injections in WT and KO induced neurons. (FIGS. 1F-1G) Representative traces and quantification of maximal spike frequency by current injection in induced WT and KO neurons. (FIGS. 1H-1I) Representative traces and quantification of spike fAHP amplitude of induced WT and KO neurons. (FIGS. 1J-1N) Induced neurons derived from AS iPSCs showed reproduced excitability and fAHP changes. (FIG. 1J) Immunostaining for UBE3A in AS neurons, with and without ectopic expression of UBE3A. Scale bar: 10 μm. (FIG. 1K) F-I curves showing spike frequency versus current injections in induced neurons derived from AS iPSCs. (FIG. 1L) Quantification of maximal spike frequency in the current injections. (FIGS. 1M-1N) Representative traces and amplitude quantification of spike fAHP. Data represent mean±SEM. Two-tailed unpaired Student's t-test for all. In all bars, n indicates the number of analyzed neurons. **p<0.01, *P<0.05, N.S. not significant.

FIGS. 2A-2I show that UBE3A deletions increase BK channel function in human neurons. (FIG. 2A) Representative traces and quantification of BK currents isolated from WT and KO neurons treated with paxilline (5 μM). (FIG. 2B) Diagrams illustrating the detection of BK channels using a functionalized probe with atomic force microscopy (AFM). (FIG. 2C) Representative heatmaps of specific BK-probe-binding events. Force-distance curves (10×10 data points) were obtained over 1 μm² areas. Shading indicates the measured force of specific binding events. (FIG. 2D) Unbinding force distribution and BK channel density on the surface of WT and KO neurons. (FIGS. 2E-2H) Pharmacological rescue by the BK antagonist paxilline. (FIGS. 2E-2F) Representative traces and quantification of fAHP with and without paxilline (5 μM). (FIGS. 2G-2H) F-I curves showing spike frequency versus current injections and related quantification in induced neurons. (FIG. 2I) dose-dependent change on fAHP amplitude upon paxilline treatment of WT human induced neurons. Data represent mean±SEM. In all bars, n indicates the number of analyzed neurons. Two-tailed unpaired Student's t-test. *** p<0.001, *P<0.05, N.S. not significant.

FIGS. 3A-3K show altered functional properties of neurons and enhanced network activity in UBE3A-deficient organoids. (FIG. 3A) Brightfield images of organoids and immunostaining of cortical layer markers in WT and KO organoids at day 120. Scale bar (brightfield images): 5 mm. Scale bar (immunostaining): 50 μm. (FIGS. 3B-3D) Altered electrophysiological properties in neurons from KO organoids. Representative traces (FIG. 3B), F-I curves evoked by current injection (FIG. 3C), and quantifications (FIG. 3D), with and without paxilline (10 μM). (FIGS. 3E-3K) Two-photon (2P) live calcium imaging of WT and KO organoids. (FIG. 3E) Calcium transient traces extracted from individual neurons of WT and KO organoids. (FIG. 3F) Cumulative distribution of inter-event-intervals (IEI) in time bins for calcium transients recorded in WT and KO organoids. Inserted bar graph shows the quantification. n=131 and 236 neurons from N=12 and 17 organoids, respectively for WT and KO. (FIG. 3G) Quantification of the amplitudes of calcium transients recorded in WT and KO organoids. (FIG. 3H) Quantification of the frequencies and amplitudes of calcium transients before and after paxilline treatment (10 μM). N=10 and 11 organoids for WT and KO, respectively. (FIGS. 3I-3J) Representative traces of calcium transients in individual neurons (left) and correlation heatmaps (right) obtained from WT (FIG. 3I) and KO (FIG. 3J) organoids. SI: synchronization index. (FIG. 3K) Summary of the synchronization index recorded in WT and KO organoids upon paxilline treatment (10 μM). N=10 and 11 organoids for WT and KO, respectively. Data represent mean±SEM. In all bars, n indicates the number of analyzed neurons. Two-tailed paired Student's t-test was used for panels H and K, Two-tailed unpaired Student's t-test was used for all others. ** p<0.01, ***p<0.001.

FIGS. 4A-4I show BK modulation ameliorates seizure susceptibility in AS mouse. (FIG. 4A) Latency to myoclonic seizure induced by flurothyl in WT and KO (Ube3a^{m-/p+}) mice. WT without paxilline, N=23; KO without paxilline, N=17; WT with paxilline, N=16; KO with paxilline, N=12. (FIG. 4B) Epilepsy grade induced by picrotoxin in WT and KO mice. WT without paxilline, N=16; KO without paxilline N=18; WT with paxilline, N=16; KO with paxilline N=16. (FIG. 4C) Samples of spectrogram and averaged delta power of LFP from BIC of WT and KO mice with sound stimuli. Delta rhythmicity below the dash line was averaged for analysis. (FIG. 4D) Summary of PSD of LFP recorded from BIC of WT and KO mice with sound stimuli. N=8 mice from 2 batches. Normalization was performed to remove the baseline difference due to batch effect. Two-way ANOVA with the Bonferroni post hoc test was used for frequencies of 1-4 Hz to analyze differences between WT and KO mice. (FIG. 4E) Mean delta (1-4 Hz) power of WT and KO mice LFPs. N=8 mice from 2 batches. (FIG. 4F) Sample spectrogram and averaged delta power of LFPs in BIC of WT and KO mice with paxilline treatment (0.35 mg/kg, 4 times with 1-hour interval). (FIG. 4G) Summary of PSD of LFPs in BIC recorded in WT and KO mice with paxilline treatment. Two-way ANOVA with the Bonferroni post hoc test was used for frequencies of 1-4 Hz to analyze differences between WT and KO mice. (FIG. 4H) Mean delta power (1-4 Hz) of WT and KO mice LFPs with paxilline treatment. N=8 mice from 2 batches. (FIG. 4I) WT with paxilline, N=5; KO with paxilline N=5. Data represent mean±SEM. Compared to 0.35 mg/kg treatment, high dosage (3 mg/kg) of paxilline induced tremor in WT mice, but this effect was not evident in KO mice. In all bars, n indicates the number of animals analyzed. Data represent mean±SEM. Two-tailed unpaired Student's t-test for all panels except for D and G. * p<0.05, ** p<0.01, N.S. not significant.

FIGS. 5A-5F show that GAL-021 application restores changes in the intrinsic excitability associated with augmented fAHP in UBE3A-deficient human neurons. (FIG. 5A) Representative traces of spikes evoked by current injection in patch clamp recordings in human induced neurons (hINs) derived from wild type (WT) and UBE3A knockout (KO) human embryonic stem cells (hESCs). (FIG. 5B) Plot of spike frequency to current inject recorded in WT and KO human induced neurons (hINs). (FIG. 5C) Summary of fAHP of first four spikes in each step of current injection in WT and KO hINs. (FIG. 5D) Representative traces of spikes evoked by current injection upon application of GAL-021 (100 μM) on WT and KO hINs. (FIGS. 5E-5F) Restoration of spike frequency and fAHP in KO neurons compared with WT hINs with GAL-021treatment (100 μM). (FIG. 5G) Dose-response relationship of fAHP amplitude upon application of various concentrations of GAL-021 (0, 50 μM, 100 μM, and 200 μM) in WT and KO hINs. Note that the application of 50 μM or 100 μM of GAL-021 did not affect fAHP amplitudes in WT hINs. In contrast, 50 μM or 100 μM of GAL-021 lowered the amplitude of fAHP in KO hINs. At 200 μM, GAL-021 lowered the fAHP amplitude in WT hINs. However, the effect of 200 μM on fAHP amplitude recorded from KO hINs was not significantly different from that of 100 μM of GAL-021 treatment. ** p<0.01, Two-way ANOVA.

FIGS. 6A-6C show the characterization of human embryonic stem cells (hESCs) and AS-patient derived iPSCs. (FIG. 6A) Normal karyotypes of hESCs and hiPSC lines used in the study. (FIG. 6B) Immunostaining with specific antibodies against pluripotency markers (OCT4 and NANOG) in hESCs and iPSCs. Scale bar: 50 μm. (FIG. 6C) Quantification of proliferating cells in WT and KO hESCs. Cells were pulse-labeled with EdU for 3 hours before immunostaining. N=4 independent batches. Data represent mean±SEM.

FIGS. 7A-7S show the molecular and functional characterization of human induced neurons. (FIG. 7A) Quantification of MAP2⁺ neurons derived from WT and KO hESCs. (FIGS. 7B-7E) Quantification of electrophysiological properties of WT and KO neurons. Cm: Capacitance; Rm: cell membrane resistance; Rinput: input resistance. (FIG. 7F) Voltage-gated sodium and potassium channel-mediated current of the induced neurons. (FIG. 7G) Representative traces to illustrate spike threshold and fAHP in patch clamp recordings. Slope of spikes (lower curve) was first calculated, and threshold was defined when slope equals to 5 mV/ms. (FIGS. 7H-7J) Normalization of the excitability and fAHP changes by ectopic expression of UBE3A in KO neurons derived from H9 hESCs. (FIG. 7H) Immunostaining for UBE3A (light grey) in WT (H9), KO, and KO neurons expressing UBE3A exogenously. Scale bar: 10 μm. (FIG. 7I) representative trace of AP. (FIG. 7J) Quantification of spike fAHP amplitude. (FIG. 7K) F-I curves showing spike frequency versus current injections in induced neurons. (FIG. 7L) Quantification of maximal spike frequency in the current injections. (FIGS. 7M-7S) H1-derived UBE3A KO neurons phenocopied neurons derived from H9. (FIG. 7M) Schematic illustrating the CRISPR-Cas9-mediated gene editing approach used to knock out UBE3A in H1 hESCs (upper) and immunoblot showing the absence of the UBE3A protein in the resultant UBE3A KO cells (bottom). (FIG. 7N) F-I curves showing spike frequency versus current injections in induced neurons. (FIG. 7O) Quantification of maximal spike frequency in the current injections. (FIG. 7P) Quantification of spike fAHP amplitude. (FIGS. 7Q-7S) Nonlinear regression sigmoid fitting of F-I curves for induced neurons derived from both H1 (2 batches) and H9 (2 batches) hESCs lines. (FIG. 7Q) Sigmoid fitting of F-I curves of induced neurons by formula f(x) using MATLAB toolbox. (FIGS. 7R-7S) Maximal spikes frequency and current injection (C.i.) for half-maximal frequency estimated from the sigmoid. *P<0.05, Two-tailed unpaired Student's t-test was used for J and K. ** p<0.01, *P<0.05, Two-tailed paired Student's t-test was used for M and N. In all bars, n indicates the number of analyzed neurons. Data represent mean±SEM.

FIGS. 8A-8F show BK augmentation in human UBE3A deficient neurons. (FIG. 8A) Representative traces demonstrating the pharmacological isolation of BK currents following the application of paxilline (5 μM). BK current was calculated by subtracting the evoked current recorded after paxilline treatment from the current evoked before paxilline treatment; voltage clamp recordings were used to perform these experiments. (FIG. 8B) Quantification of a BK current amplitude in WT and KO neurons. (FIGS. 8C-8D) Representative images and quantification of medium AHP (mAHP) in WT and KO neurons under current injection recordings. (FIG. 8E) Representative immunoblotting and (FIG. 8F) quantification of protein levels in cell lysates obtained from WT and KO human neurons. N=3 independent batches of paired WT and KO samples. *P<0.05, Two-tailed unpaired Student's t-test. N.S. not significant. In all bars, n indicates the number of analyzed neurons. Data represent mean±SEM.

FIGS. 9A-9C show a schematic illustrating AFM-based BK probing. biochemical analyses of the effect of UBE3A expression on BK levels. (FIG. 9A) Schematic of the functionalization of the AFM tip with a BK antibody. (FIG. 9B) Diagrams illustrating the detection of BK channels using a functionalized probe with AFM. Force-distance curves obtained in the presence and absence of BK channels. CM: cell membrane. (FIG. 9C) Representative force-distance curves observed under the conditions indicated.

FIGS. 10A-10D show data on neuronal excitability and fAHP changes in human neurons upon various drug treatments. Summary of F-I curves, maximum spike frequencies, and fAHP amplitudes with paxilline treatment (5 μM) on neurons induced from (FIG. 10A) H1 hESCs, WT and KO; and (FIG. 10B) from AS-iPSC line, WT: AS-iPSC+UBE3A; KO, AS-iPSC. WT and UBE3A-KO neurons were treated with (FIG. 10C) IBTX (100 nM) and (FIG. 10D) apamin (200 nM). ** p<0.01, *P<0.05, Two-tailed unpaired Student's t-test. N.S. not significant. In all bars, n indicates the number of analyzed neurons. Data represent mean±SEM.

FIGS. 11A-11I shows UBE3A-mediated ubiquitination and degradation of BK. (FIG. 11A) Coimmunoprecipitation assay of BK and UBE3A performed by overexpressing Flag-tagged BK (Flag-BK) and HA-tagged UBE3A (HA-UBE3A) in human embryonic kidney (HEK293) cells. (FIG. 11B) Ubiquitination assay of BK performed by coexpressing HA-tagged ubiquitin (HA-Ub) and wildtype (WT) or a catalytically dead mutant of UBE3A (MT, C833A) in HEK293 cells. (FIG. 11C) In vitro ubiquitination assay of BK and UBE3A. E1: Ube1, E2: UbcH5c, HA-Ub: HA tagged Ubiquitin. (FIGS. 11D-11E) Endogenous BK ubiquitination in primary mouse cortical neurons. BK was immunoprecipitated (IP) and BK ubiquitination was detected by Ubiquitin antibody. N=3 batches. Intensity was normalized to WT for each batch. Data represent mean±SEM. Two-tailed unpaired Student's t-test. *** p<0.001. (FIG. 11F) Immunoblotting illustrating changes in BK protein levels after exogenous expression of increasing amounts of WT UBE3A in HEK293 cells. WT: wild-type UBE3A. (FIGS. 11G-11H) Representative western blots and quantification of BK levels in HEK293 cells overexpressing WT or MT UBE3A treated with or without MG132 (1 μM). *P<0.05 (one-way ANOVA). (FIG. 11I) Immunoblotting illustrating changes in BK protein levels in HEK293 cells overexpressing WT UBE3A or mutants (T485A, E550L, and L502P).

FIGS. 12A-12D show qPCR and RNA-sequencing analyses of human induced neurons. (FIG. 12A) Quantification of mRNA levels of channel genes based on quantitative RT-PCR. N=4 independent batches of samples. Data represent mean±SEM. (FIG. 12B) Quality control of RNA-seq libraries. Percentages and absolute quantities of reads uniquely mapped to the human genome after the removal of rat reads. M=Millions of reads. (FIG. 12C) Principal component analysis (PCA) performed before and after batch effects were modeled, showing clustering by genotype along the first principal component (PC1) after correction. (FIG. 12D) Volcano plot showing the differential expression analysis of UBE3A KO vs WT RNA-seq data. FDR: False discovery rate-adjusted p-value.

FIGS. 13A-13B show RNA sequencing analyses of human induced neurons. (FIG. 13A) Gene set enrichment analysis for Gene Ontology categories using differentially expressed genes (DEGs) between KO and WT induced neurons. (FIG. 13B) Normalized read counts for BK channel related genes: KCNMA1 for BKα, KCNMB1 for BKβ1, KCNMB2 for BKβ2, KCNMB3 for BKβ3, and KCNMB4 for BKβ4. FDR=False Discovery Rate-adjusted p-value for differential expression.

FIGS. 14A-14D show characterization of human cortical organoids. (FIGS. 14A-14B) Representative images of immunostaining with neural progenitor markers and quantification in WT and KO organoids on day 20. Scale bar: 80 μm. (FIG. 14C) Quantification of cortical neuron markers in WT and KO organoids on day 120. Data represent mean±SEM. (FIG. 14D) Representative images of organoids immunostained with antibodies against GABA and GFAP. Scale bar: 20 μm.

FIGS. 15A-15K show functional characterization of brain organoids derived from WT, KO, and AS iPSCs. (FIGS. 15A-C) Quantification of electrophysiological properties of neurons in the organoids. Cm: Capacitance; Rinput: input resistance; RMP: resting membrane potential. (FIG. 15D) Immunoblotting of BK protein and other neuronal markers in WT and UBE3A-KO organoids. (FIGS. 15E-15G) F-I curve and quantification of spike frequencies and fAHP amplitudes in organoid neurons generated from AS-iPSCs and AS-iPSCs overexpressing UBE3A. (FIG. 15H) Spontaneous calcium spikes before and after TTX treatment (2 μM). **P<0.01, *P<0.05, Two-tailed paired Student's t-test was used for all panels. (FIG. 15I) Calcium transients evoked by graded electrical trains-stimuli (Stim.) and recorded by 2P imaging. (FIG. 15J) Representative traces of spontaneous spikes observed in perforated patch clamp recordings obtained from KO organoids before and after paxilline treatment (10 μM). (FIG. 15K) Quantification of spontaneous burst frequencies observed before and after paxilline treatment. Spike trains with more than 4 spikes were considered as bursts. N=4 organoids. Data represent mean±SEM.

FIGS. 16A-16D show BK augmentation and increased fAHP in neurons of Ube3a^{m-/p+} mice. (FIG. 16A) Schematic of Ube3a deletion in the maternal allele in Ube3a^{m-/p+} mice (top) and Ube3a loss confirmed by immunoblotting (bottom). (FIG. 16B) BK current isolated from acute hippocampal slices of WT and KO mice by paxilline (10 μM). Quantification of fAHP amplitudes (FIG. 16C) and maximal spike frequency with current injections (FIG. 16D) of recorded mouse neurons. In all bars, n indicates the number of analyzed neurons. Data represent mean±SEM.

FIGS. 17A-17B show LFP recordings in the brain of WT and Ube3a^{m-/p+} mice. (FIG. 17A) Schematic of local field potential (LFP) recording in the brachium of inferior colliculus (BIC) with audio stimuli (˜125 dB). Raw LFPs (mid) were recorded by sampling at 500 Hz, filtered with 0.5 Hz high-pass and 100 Hz low-pass, and then analyzed by fast Fourier transform (FFT) for power spectral density (PSD). (FIG. 17B) Quantification of different rhythmicity power of LFP recorded from WT and Ube3a^{m-/p+} mice. Theta: 4-8 Hz; Beta: 12-30 Hz; Gamma: 30-80 Hz. Data represent mean±SEM.

### DETAILED DESCRIPTION OF THE INVENTION

### Definitions

Certain terms employed in the specification, examples and appended claims are collected here for convenience.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

The term “subject” is herein defined as vertebrate, particularly mammal, more particularly human. For purposes of research, the subject may particularly be at least one animal model, e.g., a mouse, rat and the like. In particular, for treatment or prophylaxis of a UBE3A channelopathy, such as Angelman Syndrome, the subject may be a human.

The term “treatment”, as used in the context of the invention refers to ameliorating, therapeutic or curative treatment.

Bibliographic references mentioned in the present specification are for convenience listed in the form of a list of references and added at the end of the examples. The whole content of such bibliographic references is herein incorporated by reference.

Having now generally described the invention, the same will be more readily understood through reference to the following examples which are provided by way of illustration, and are not intended to be limiting of the present invention.

A person skilled in the art will appreciate that the present invention may be practiced without undue experimentation according to the methods given herein. The methods, techniques and chemicals are as described in the references given or from protocols in standard biotechnology and molecular biology text books.

### EXAMPLES

### Example 1

### General Methods

hPSCs Cell Culture

Human ESC lines H1 and H9 were originally obtained from WiCell Research Institute (Madison, Wis.), and have been maintained in the laboratory. AS iPSC was obtained from Kerafast (AG1-0). All hESC and hiPSC lines were cultured in mTeSR1 media (Stemcell Technologies) under feeder-free conditions in matrigel-coated cell culture plates and were routinely passaged (1:100) using Versene (Thermofisher). All lines displayed normal karyotypes.

### Animals

Transgenic mice used in present study were originally generated by the Beaudet's lab and backcrossed more than 10 generations with C57BL/6J (Jiang et al 1998a). We obtained the mice from the lab of Yong-Hui Jiang (Duke University, USA). All experiments related to animals were conducted following the protocol approved by the Institutional Animal Care and Use Committee (IACUC) of SingHealth. Mice were housed in SPF rooms with a 12-hour light/dark cycle. Wild-type (Ube3a^{m+/p+}) and maternal knockout (Ube3a^{m-/p+}) mice were produced from breeding of Ube3a^{m+/p-} females and Ube3a^{m+/p+} males. Genotyping was conducted by using primers:

P1:
SEQ ID NO: 1;
5′-AAC TTC ACT GCT ACA TCT CCA TGG C;
P2:
SEQ ID NO: 2;
5′-GCT CAA GGT TGT ATG CCT TGG TGC T;
P3:
SEQ ID NO: 3.
5′-TGC ATC GCA TTG TCT GAG TAG GTG TC;

Generation of UBE3A KO hESCs

A single guide RNA (sgRNA) was designed using the online Benchling design tool (https://benchlingdotcom), targeting exon 6 of human UBE3A gene. sgRNA sequence is: 5′-CTACTACCACCAGTTAACTGAGG-3′ (SEQ ID NO: 20) and was cloned into lenti-CRISPR v2 plasmid (Addgene, #52961) as previously described (Sanjana et al., Nat Methods 11, 783-784 (2014)). The Cas9-gRNA construct was isolated using E.Z.N.A. maxi kit (OMEGA). 5 μg of Cas9-sgRNA expression plasmid were used to transfect into human ESCs (1×10⁶ cells) using nucleofection kit (Lonza). Next day, cells were selected by puromycin (500 ng/ml) for 2 days, followed by dissociation into single cells and were replated at low density on a matrigel-coated plate with mTeSR1 medium (Stemcell Technologies). Single hESC clones were picked and split into 2 batches (one for genotyping and the other for maintenance). Targeted UBE3A locus were amplified using REDiant Taq DNA polymerase (Fist base, Bio-5115-500U). PCR products were analysed by Sanger sequencing.

Generation of Induced Neurons from hPSCs

hPSCs were induced to neurons as previously described (Sun et al., Cell Rep 16, 1942-1953 (2016); Zhang et al., Neuron 78, 785-798 (2013)). Briefly, hESCs and hiPSCs were dissociated with TrypLE Express (Thermofisher) to single cells and plated onto matrigel coated cell culture plates in mTeSR1 media supplemented with thiazovivin (1 μM, Tocris). Next day, cells were transduced with lentiviruses expressing tet-mNGN2 and rtTA. On the following day, culture media was completely replaced with Neuronal Media (Sciencell) supplemented with doxycycline (1 μg/ml) for the next 5 days. Cells were selected with puromycin (3 μg/ml) for 48 hours starting day 3 to enrich for transduced cells. At day 5, cells were dissociated to single cells by TrypLE Express (Thermofisher) and replated onto either matrigel-coated glass coverslips (for immunostaining, AFM, and electrophysiological recordings) or 10 cm cell culture plates (for biochemical assays) in BrainPhys media (Stemcell Technologies) supplemented with SM1 (1×, Stemcell Technologies) and Antibiotic-Antimycotic (1×, Thermofisher). Primary rat glial cells were added onto human induced neuronal cultures at day 7. Neurotrophic factors (BDNF, GDNF, NT3, each at 10 ng/ml, all from PeproTech) and 1% FBS and FUdR (5-fluorodeoxyuridine: 16.5 mg/ml, Sigma F0503 with Uridine: 6.7 mg/ml, Sigma U3003) were added starting day 10 until the day of analyses.

Generation of Cortical Organoids from hPSCs

Cortical organoids were generated based on a published protocol (Pasca et al., Nat Methods 12, 671-678 (2015)) with minor modifications. hPSCs were dissociated to single cells by TrypLE Express (Thermofisher) and seeded into U-shaped ultra-low attachment 96-well plate (Corning) at a density of 6K cells/well in the presence of thiazovivin (5 μM, Tocris). After spheroid formation, media were completely changed to Neural Induction Media (NIM: DMEM/F12 (Nacalai Tesque), 20% KnockOut Serum Replacement (Thermofisher), 1% minimum essential media-nonessential amino acid (Thermofisher), 1% GlutaMax (Thermofisher), 1% Antibiotic-Antimycotic (Thermofisher), 0.1% β-mercaptoethanol (Thermofisher), 5 μM Dorsomorphin (Tocris), 10 μM SB431542 (Stemgent)) for the next 6 days, with media change every other day. Starting day 7, media were changed to Neural Growth Media (NGM: Neurobasal (Thermofisher), 1:50 B27 without vitamin A (Thermofisher), 1% GlutaMAX (Thermofisher), 1% Antibiotic-Antimycotic (Thermofisher), 20 ng/ml EGF (PeproTech), 20 ng/ml FGF-basic (PeproTech)) for the next 18 days, with media change every other day. Starting day 25, organoids were transferred out of 96-well plates to 6-well plates (6-8 organoids per well), cultured in Organoid Media I (OMI: Neurobasal (Thermofisher), 1:50 B27 without vitamin A (Thermofisher), 1% GlutaMax (Thermofisher), 1% Antibiotic-Antimycotic (Thermofisher), 10 mM HEPES (Thermofisher), 10 ng/ml BDNF (PeproTech), 10 ng/ml NT3 (PeproTech)) until day 100 on an orbital shaker (75 rev/min). After day 100, media were changed to Organoid Media II (OMII: BrainPhys (Stemcell Technologies), 1:50 SM1 (Stemcell Technologies), 1% GlutaMax (Thermofisher), 1% Antibiotic-Antimycotic (Thermofisher), 10 mM HEPES (Thermofisher), 10 ng/ml BDNF (PeproTech), 10 ng/ml GDNF (PeproTech), 10 ng/ml NT3 (PeproTech), 100 μM db-cAMP (Sigma)).

### Production of the 3rd Generation Lentiviral Particles

To generate lentiviral particles, lentiviral expression vectors (tetO-NGN2-Puro: Addgene #52047; FUW-rtTA: Addgene #20342) together with helper plasmids (pMDLg/pRRE: Addgene #12251; pRSV-Rev: Addgene #12253; pMG2.G: Addgene #12259) were co-transfected into Lenti-X 293T cells (Clontech) using Fugene HD (Roche). Supernatants were collected from culture media and lentiviral particles were concentrated using Lenti-pac concentration solution (GeneCopoeia).

### Gene Expression Analyses

For quantitative RT-PCR analyses of human neurons, total RNA was extracted using DirectZol (Zymo), treated with DNAse, and converted to cDNA using High-Capacity cDNA Reverse transcription kit (Life Technologies). Real-time PCR assay was performed using the Applied Biosystems 7900HT Fast real-time PCR system. Primers sequences used in the study was included in Table 1.

TABLE 1
Sequence information of primers used in quantitative real time RT-PCR

| Gene | Forward (5′-3′) | SEQ ID | Reverse (5′-3′) | SEQ ID |
|---|---|---|---|---|
| GAPDH | TTGAGGTCAATGAAGGGGTC | 4 | GAAGGTGAAGGTCGGAGTCA | 5 |
| MAP2 | CAACGGAGAGCTGACCTCA | 6 | CTACAGCCTCAGCAGTGACTA | 7 |
| BK α | ACAAGTCTGCCAACCGAGAG | 8 | ATGGACATCTTGGGCCGTTT | 9 |
| Na⁺/K⁺- | TGCTCTGTGCTTTTCTCTCTG | 10 | TTCAGTTCATCCATGTCCCTG | 11 |
| ATPase α1 | | | | |
| Na⁺/K⁺- | GCGTGTGCTGGGATTCTGTC | 12 | TGGCCTTGGCTGTGATAGGG | 13 |
| ATPase α2 | | | | |
| Na⁺/K⁺- | AACCTAAGCCTCCCAAGAATG | 14 | CAGGAGTTTGCCATAGTACGG | 15 |
| ATPase β1 | | | | |
| Na⁺/K⁺- | TTCGTGTGGAACCCGAGGAC | 16 | GCGAATCATCAAGCCCGGTG | 17 |
| ATPase β2 | | | | |

### Fluorescent Immunocytochemistry

hESCs or induced neurons were fixed with 4% paraformaldehyde (PFA) in PBS for 15 minutes, permeabilized with 0.25% triton X-100 in PBS for 15 minutes and blocked by blocking buffer (5% BSA and 1% FBS in PBS) for 60 mins. Next, samples were incubated with primary antibodies (chicken anti MAP2: Abcam AB5392, rabbit anti Synapsin I: SYSY 106103, mouse anti UBE3A: SIGMA E8655, rabbit anti UBE3A: SIGMA HPA039410) 2 hours in room temperature (RT) or overnight at 4° C. Samples were incubated with secondary antibodies for 1 hour in RT and mounted on glass coverslips. Images were acquired using an LSM 710 (Zeiss) confocal microscope. Quantification (synaptic boutons, neuronal complexities, and etc.) were done using a MetaMorph software.

Organoids were fixed in 4% PFA in PBS overnight, washed in PBS, incubated in 30% sucrose in PBS at 4° C. overnight, and subsequently embedded in O.C.T. (Sakura Finetek). Fixed organoid samples were cryosectioned using a cryostat (Leica). For immunofluorescence, cryosections were washed with PBS to remove excess O.C.T compound and blocked with 3% BSA and 0.5% Triton X-100 in PBS for 1 hr at RT. Sections were incubated with diluted primary antibodies overnight at 4° C. and secondary antibodies for 1 hr at RT. All sections were counterstained with DAPI (Sigma-Aldrich) and mounted with FluorSave media (Millipore). Images were taken on an LSM 710 (Zeiss) or TCS SP8 (Leica) confocal microscopes. The following primary antibodies were used: chicken anti MAP2 (Abcam AB5392), mouse anti NeuN (Millipore MAB377), rabbit anti BRN2 (GeneTex GTX114650), rabbit anti TBR2 (Abcam AB23345), rat anti CTIP2 (Abcam AB18465), mouse anti CUX1 (Abnova H00001523-M01), rabbit anti GABA (Sigma A2052), mouse anti GFAP (Milipore MAB360).

### Electrophysiology

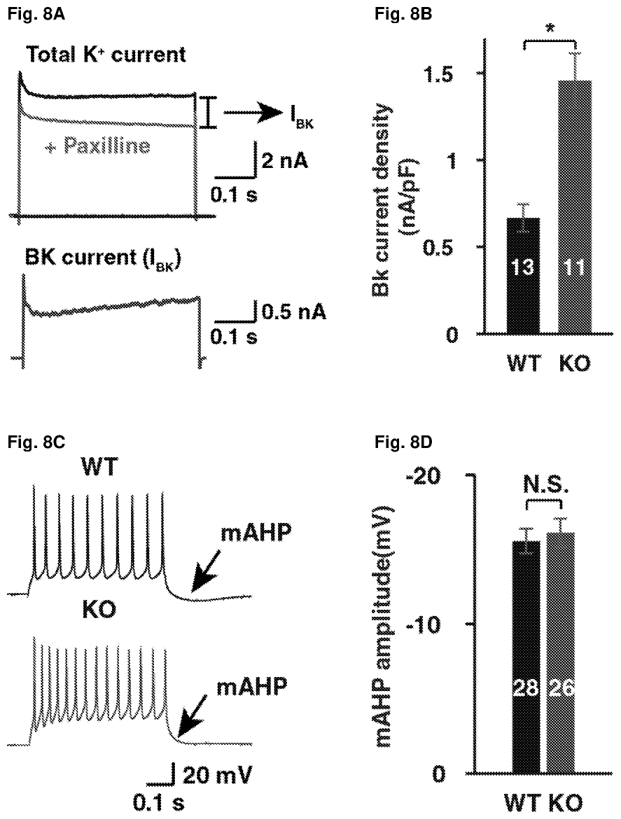

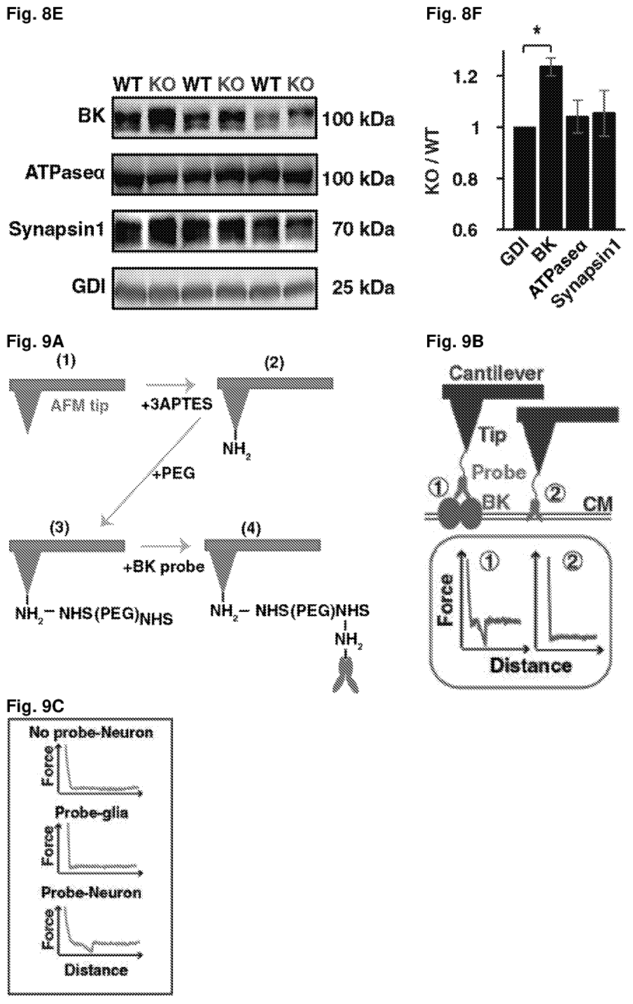

Whole cell patch clamp recordings were performed on induced neurons in RT similar as described previously (Yi et al., Science 352, aaf2669 (2016)). Recording pipettes with resistances of 4-6 MΩ were filled with internal solution containing (in mM): 135 KMeSO₃, 10 KCl, 1 MgCl₂, 10 HEPES, 2 Na₂ATP, 0.4 Na₃GTP with osmolarity of 290 mOsm and pH 7.3˜7.4. Neurons were bathed in the extracellular solution containing (in mM): 140 NaCl, 3 KCl, 10 dextrose, 2 MgCl₂, 3 CaCI₂, 10 HEPES at pH 7.3-7.4. Neuronal intrinsic excitability was measured in the presence of CNQX (20 μM, Tocris) and APV (50 μM, Tocris) to block all excitatory synaptic responses. Recording was sampled at 40 kHz and filtered at 2 kHz (Digidata 1440A, Molecular Devices). We only chose pyramidal-shaped, similar-sized neurons based on their capacitance measurements. Data with serial resistance higher than 20 MΩ or leaking current more than 200 pA were rejected. Resting membrane potential was estimated in current-clamp mode immediately after breaking into the membrane and establishing whole-cell configuration. For analysis of the parameters of evoked action potentials, stepped current injections (500 ms duration, 20 pA stepwise from −20 pA to +360 pA with 4 s interval) were performed using the current clamp model. Stepped voltage depolarization (500 ms duration, 20 mV stepwise and 10 steps with 4 s interval) was performed to obtain voltage-gated sodium and potassium currents as well as pharmacological isolation of BK currents (FIG. 8A).

Whole cell patch clamp recordings of neurons in the organoid (wholemount) and acute mouse hippocampal slices were performed in a recording chamber (RC-26GLP, Warner Instruments) and submerged under continuously perfused artificial CSF (aCSF) containing (in mM): 119 NaCl, 2.5 KCl, 11 glucose, 26 NaHCO₃, 1.25 NaH₂PO₄, 2.5 CaCl₂ and 2 MgCl₂ saturated with 95% O₂ and 5% CO₂ at 30-32° C. Neuronal intrinsic excitability was measured in the presence of CNQX (20 μM, Tocris) and APV (50 μM, Tocris) to block all excitatory synaptic responses. We targeted neurons near the surface of organoids with reasonably pyramidal-shaped cell bodies under our DIC/IR camera. Patch glass pipettes (3-4 MΩ) were filled with an intracellular solution containing (in mM): 135 KMeSO₃, 10 KCl, 10 HEPES, 1 EGTA, and 2 Na₂ATP. The osmolarity was adjusted to 290 mOsm, and the pH was adjusted to 7.3-7.4. The whole cell recordings were performed using an Olympus BX51WI upright microscope equipped with a 60× water-immersion lens with DIC-IR. Signals were recorded using a MultiClamp 700B amplifier, filtered at 2 kHz using a Bessel filter, and digitized at 40 kHz with a Digidata 1550B analog-to-digital (A/D) board (Molecular Devices, Sunnyvale, Calif.). Resting membrane potential was estimated in current-clamp mode immediately after breaking into the membrane and establishing whole-cell configuration. To test neuronal excitability, a series of current pulses (500 msec) of increasing amplitude from 0 pA to +35 pA (in 5 pA increment, 4 s interval) for organoid neurons and from 0 pA to +240 pA (in 10 pA increment, 4 s interval) for mouse neurons, respectively, were injected to obtain the frequency-current injection (F-I) curve.

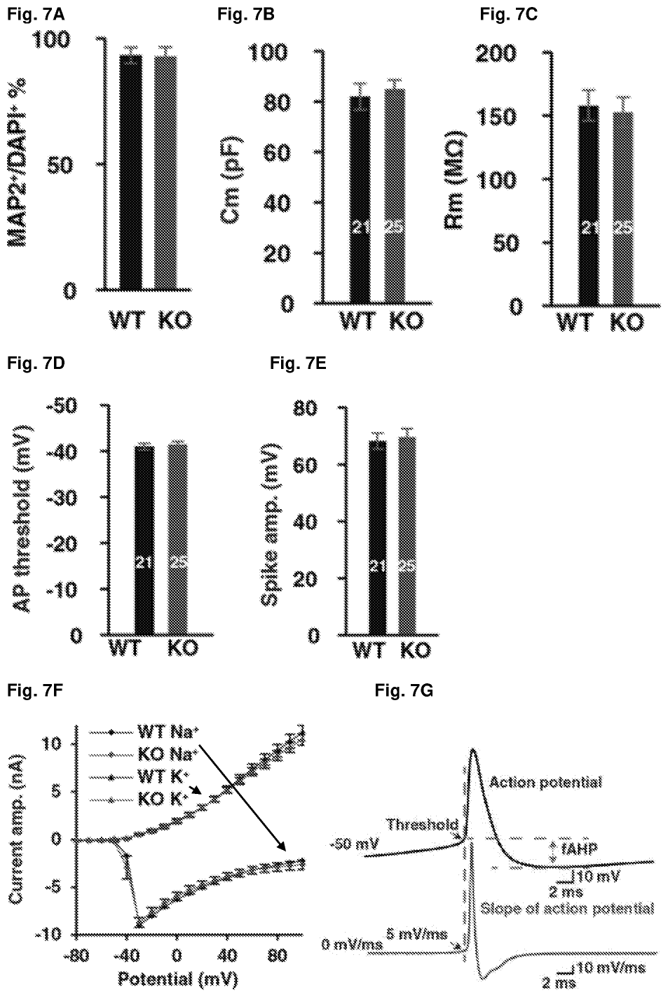

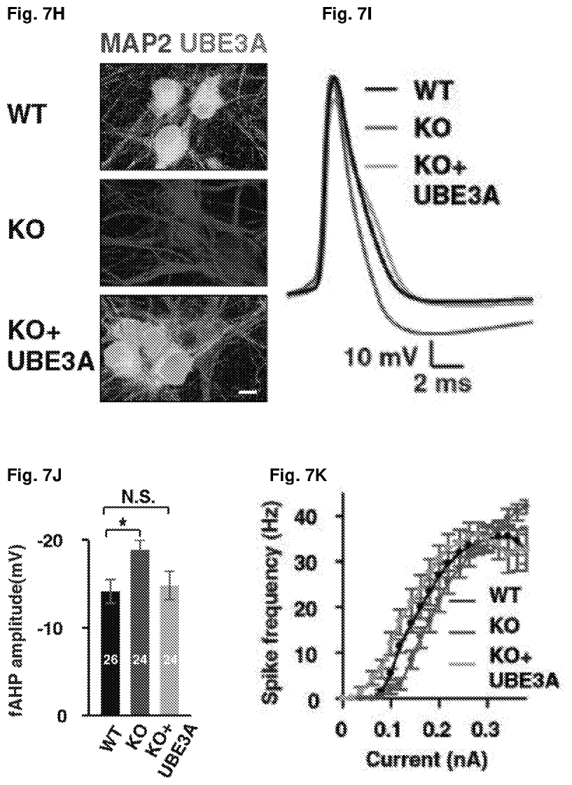

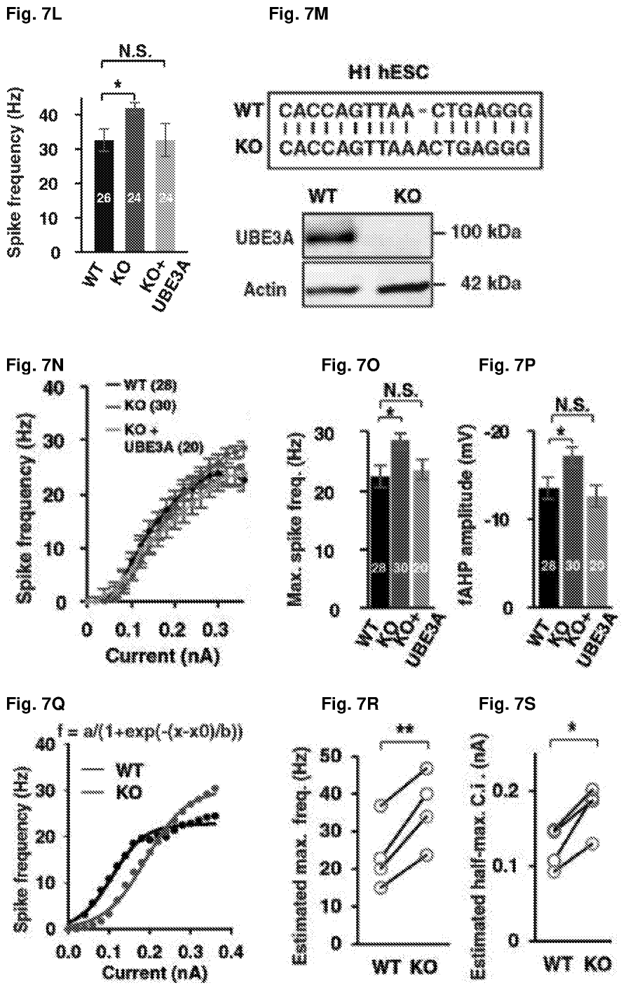

For fAHP analysis in all recordings, automatic detection and calculation was conducted by customized matlab scripts adapted from published algorithm (Bean, Nat Rev Neurosci 8, 451-465 (2007); Platkiewicz and Brette, PLoS Comput Biol 6, e1000850 (2010); Yu et al., J Neurosci 28, 7260-7272 (2008)). Spikes threshold was defined as the time-point when membrane potential increasing slop equal to 5 mV/ms. The amplitude of the fast AHP (fAHP) was defined as the voltage difference between spike threshold and the lowest point in each given spike within 10 ms (FIG. 7G). The fAHP amplitude of each neuron was calculated by averaging the fAHP of the first spike in all current injection steps. We ran automated scripts to detect the threshold of action potentials and fAHPs in a blind-manner.

### In Vivo Local Field Potential (LFP) Recording in Freely Moving Mice.

Mice (10-12 weeks) were anesthetized with continuous isoflurane at the stereotaxic apparatus (RWD). Eye cream was applied to a mouse to protect the eye from drying due to anaesthesia. 2% lidocaine was injected under the scalp and subsequently scalp was removed to expose the skull. The brachium of inferior colliculus (BIC) was located by the following coordinate: anteriorposteror (AP), −4.1 mm; Lateral, 2.1 mm; Depth, 2 mm, referring from Bregma. Stereotrodes were made with platinum-iridium wire (90%:10%, A-M system) and unilaterally implanted into BIC. Stainless steel screw (AP: 2 mm, Lateral: 2 mm) was used as a reference electrode. Iodopavidone was applied for disinfection around the surgical area after dental cement mounting. Mice were maintained on a heating pad at 37° C. until waking from the anaesthesia before putting back to home cages. LFP recordings were performed on the mice 5 days after surgery. LFP recordings were conducted in a copper-Faraday chamber. For evoked LFP, auditory stimulus (˜125 dB, 500 Hz) was provided by a speaker (EKX, Electro-Voice) driven by a customized program in Matlab. LFP was recorded with sampling rate at 500 Hz and filtered with 0.5 Hz high-pass and 100 Hz low-pass by EEG/EMG recording system (Pinnacle). Raw signals were converted by fast Fourier transform (FFT) to calculate power spectral density (PSD) using Matlab and NeuroExplorer. Video was recorded simultaneously for further analysis. After all recordings, mice were sacrificed, and their brains were isolated and fixed using 4% PFA in PBS and the implanted electrode locations were confirmed by either bright field imaging or DAPI staining and subsequent fluorescence imaging.

### Biochemistry

Induced neurons or cortical organoids were lysed in RIPA buffer supplemented with protease and phosphatase inhibitor cocktails. Protein samples were loaded onto 8-12% Bis-TRIS (NP0322BOX Invitrogen) and were transferred onto a PVDF membrane. After blocking with 5% milk in TBS-T buffer for 1 hour, PVDF membranes were incubated with primary antibodies overnight at 4° C., followed by 1-hour incubation with mouse or rabbit HRP antibody (1:2000, Invitrogen) at RT. Images were visualized by either chemiluminescence or NIR fluorescent detection using c600 Imaging System (Azure Biosystems Inc). HEK293 cells were grown in DMEM (Sigma) containing 10% FBS (Gibco) and 100 U/mL penicillin-streptomycin (Gibco) in a 5% CO₂ incubator. Cells were transfected with a Lipofectamine PLUS (Invitrogen), subsequently were treated with 1 μM of MG132 (A.G Scientific) for another 24 h. Next, cells were lysed in PBS containing 1% SDS (Invitrogen), 1 mM PMSF (USB), 10 μg/ml aprotinin (Roche), and 1% (v/v) phosphatase inhibitor cocktail 2 and 3 (Sigma).

Co-IP in HEK293 Cells: HEK293 cells were transfected with either Flag-BK (Gift of Dr. Cang Yong, ZJU, China) and/or HA-UBE3A plasmid (Addgene #8658) using lipofectamine 3000 (ThermoFisher) and lysed in a buffer containing 1% Nonidet P-40, 150 mM NaCl and 50 mM Tris-HCl with 100 μM CaCI₂, 1× protein inhibitor (Roche), and 1×PMSF. Lysates were centrifuged for 15 mins at 4° C. after 20-min incubation on ice. Collected supernatant was pre-washed with 50 μl mouse-IgG beads for 2 hours and subsequently incubated with 25 μl of FLAG-M2 agarose beads (A2220, SIGMA) overnight at 4° C. Beads were collected via brief centrifugation, washed, and boiled with LDS sampling buffer (Invitrogen) for western analysis.

In vivo ubiquitination: HEK293 cells were transfected with HA-ubiquitin (K63R), Flag-BK plasmids with either myc-UBE3A-WT or myc-UBE3A-MT plasmid with lipofectamine 3000. MG132 (1 μM) was added in the culture. Cells were collected 24 hours after transfection and subsequently lysed in a buffer containing 1% Nonidet P-40, 150 mM NaCl, 50 mM Tris-HCl and 5 μM iodoacetamide. Lysates were centrifuged at 13,500 rpm for 15 mins at 4° C. after 20-min incubation on ice. Next, supernatants (400 μg of total protein) were used for each condition and these supernatants were incubated with 40 μl of FLAG-M2 agarose beads (A2220, sigma) overnight in a continuously rotating platform at 4° C. Agarose beads were collected via brief centrifugation, washed with lysis buffer, and boiled with LDS sample buffer (NP0007, Invitrogen) for subsequent western analysis.

In vitro ubiquitination: HEK293 cells were transfected with BK expression constructs and lysed in a radioimmune precipitation assay buffer containing 1% Nonidet P-40, 0.1% SDS, 0.5% sodium deoxycholate, 150 mM NaCl, 1 mM PMSF, 10 μg/ml aprotinin and 1% (v/v) phosphatase inhibitor cocktail 2 and 3 (SIGMA) in PBS. Lysates were centrifuged, and BK proteins were immunoprecipitated with anti-FLAG M2 agarose beads. These beads were then resuspended in an assay buffer provided by the ubiquitination kit K-230 (Boston Biochem). In vitro ubiquitination was performed according to the manufacturer instructions.

Endogenous BK ubiquitination: Primary cortical neuronal cultures were used to assess endogenous ubiquitination of BK. Fast genotyping was done before the preparation of neuronal cultures. Briefly, tails of newborn pups (2-3 mm) were collected and lysed by Tween-20 based lysis buffer with proteinase K at 55° C. for 20 min. Fast PCR was completed with protocol: denaturation (98° C., 7 s)-annealing (60° C., 10 s)-extension (72° C., 15 s) for 30 cycles. Mice brains of the same genotype were pooled. Cortices were first dissected out and dissociated to single cells using Trypsin (0.25%, Invitrogen) supplemented with DNAase I (1 mg/ml, Sigma). Cells were plated to 10 cm dishes pre-coated with poly L-lysine (Sigma), and maintained in neuronal media consisting of Neurobasal medium, 1× B27 supplement, 1× Glutamax, and 1× penicillin-streptomycin (all from Invitrogen). At days in vitro (DIV) 13, neurons were treated with MG132 (1 μM) for 18-24 hours. At DIV14, cells were lysed in lysis buffer containing 1% Nonidet P-40, 150 mM NaCl, 50 mM Tris-HCl, 50 mM NaF and 5 μM iodoacetamide. BK antibody (4 μg, Alomone) and protein-A/G beads was added to the lysates and incubated overnight at 4° C. Next day, beads were collected and boiled with LDS sample buffer before western blotting. Antibodies were used: rabbit-anti-BKα (Alomone, APC151), rabbit-UBE3A (SIGMA, E8655), rabbit-UBE3A (SIGMA, HPA039410), rabbit-Flag (SIGMA, F7425), rabbit-HA (SIGMA), goat-HA (SIGMA), rabbit-Myc (Millipore)

### Atomic Force Microscopy (AFM)

A BK-channel specific antibody targeting an extracellular epitope (APC151, Alomone) was conjugated on a functionalized AFM tip (shown in FIGS. 9A and 9B). Briefly, silicon nitride cantilevers (AFM tips) were dried after rinsing with ethanol and ultrapure water and were incubated in 4% 3-aminopropyltrimethylethoxysilane (3-APTES) for 2 hours at RT. Next, tips with attached NH2 were incubated with a NHS-PEG-NHS linker (2 mg/ml in methylene chloride, Invitrogen) for 2 hours and were subsequently incubated with a BK-channel specific antibody (20 μg/ml) in PBS for 2 hours in RT. Functionalized tips were treated with further 1 mg/ml glycine in PBS to quench free functional groups. Control tips were prepared by following the same protocol without antibody conjugation.

AFM force measurements were performed as previously reported (Li et al., PLoS One 6, e16929 (2011); Lim et al., Sci Rep 7, 4208 (2017)). Silicon AFM probes with a nominal spring constant of 0.03 N/m and a tip radius of 10 nm (MSCT-D, Bruker) were used in a Nanowizard II BioAFM (JPK instruments AG, Germany). Functionalized cantilevers were calibrated in situ by a non-destructive thermal tune method using the build-in function provided by the JPK SPM Control Software. Neurons were fixed with 4% PFA in PBS and blocked by 5% BSA in PBS for 30 mins before AFM measurements (neurons with reasonably pyramidal-shaped cell bodies were selected for AFM measurement). A maximum loading force of 0.1 nN, Z length of 5 μm and a constant speed of 2.0 μm/s were used to generate single-peak specific interaction force curves (single bond rupture) (shown in FIG. 9C). Force mappings were carried out in a 1 μm² scan area (with 10×10 data points) and at least 3 random locations were probed in each cell. JPKSPM data processing software v.5 was used to analyse the raw data obtained from the aforementioned protocol. Control experiments were conducted to eliminate potential artifacts resulting from nonspecific interactions, and to ensure that the measured forces resulted from specific interactions between cantilever coated receptors and the BK channels on cell surface. Probes without antibody were tested on WT neurons and functionalized probes were tested on glial cells (FIG. 9C).

### RNA Sequencing

Human neuron-rat astrocyte co-cultures at day 40 were lysed in TRIzol (Thermofisher) and total RNAs were extracted using a Direct-zol RNA miniPrep kit (Zymo Research). RNA integrity was verified using an Agilent RNA 6000 Nano Chip and samples with a RIN above 8 (Bioanalyzer, Agilent) were further processed. cDNA libraries were prepared using Truseq Stranded mRNA (Poly A selection) kit (Illumina) and sequenced on a Hisea3000 platform (Illumina) in paired-end mode. Sequenced libraries were trimmed using cutadapt (v. 1.13) software with parameters --quality-base=33 -m 20 and adaptor pairs AGATCGGAAGAGCACACGTCTGAACTCCAGTCA (SEQ ID NO: 18) and AGATCGGAAGAGCGTCGTGTAGGGAAAGAGTGT (SEQ ID NO: 19). Trimmed reads were processed using Xenome (v. 1.0.0) software separating rat reads (aligning to R. norvegicus release Rnor_6.0) and human reads (aligning to H. sapiens genome release GRCh38.p10). Reads classified by Xenome as “human”, “both” and “ambiguous” were merged together and carried forward in the analysis. Human reads were aligned again to the same genome using STAR (v. 2.5.2b) software with the following parameters: --outFilterMultimapNmax 20 --alignSJoverhangMin 8 --alignSJDBoverhangMin 1 --outFilterMismatchNmax 999 --alignIntronMin 20 --alignIntronMax 1000000 --alignMatesGapMax 1000000 (Conway et al., Bioinformatics 28, i172-178 (2012); Dobin et al., Bioinformatics 29, 15-21 (2013)). Only uniquely mapping reads were used for counting.

Read counts were obtained at the gene level using featureCounts (v. 1.5.1) software with the Ensembl release 92 human GTF and the following parameters: -t gene -g gene_id -O -s 2 -J -T 8 -p -R. Raw counts were imported in R (v. 3.4.3) software (Liao et al., Nucleic Acids Res 41, e108 (2013)). Counts were normalized (DESeq2 between-sample normalization), log-transformed and principal component analysis was performed to visualize any confounding effects. Batch effect removal was simulated using the combat function in the software Bioconductor package sva 3.32.1 (Leek et al., 2019), showing that accounting for the replicate effect in the linear model correctly clusters samples by genotype along the first principal component. Differential gene expression analysis was performed with DESeq2 (v. 1.18.1) software using the formula ˜replicate+condition and the Wald test (Leek et al., Bioinformatics 28, 882-883 (2012); Leek and Storey, Proc Natl Acad Sci USA 105, 18718-18723 (2008); Love et al., Genome Biology 15, 550 (2014)). Reported fold changes have been moderated using the IfcShrink function in DESeq2, setting an alpha value of 0.05 for independent filtering and using default parameters. Nominal p-value distributions were checked by plotting their frequency histogram, revealing a pathological underestimation of the variance of the null distribution by the Wald test. The variance was empirically estimated and P-values were recalculated using the R package fdrtool v 1.2.15 software (Strimmer, K. BMC Bioinformatics 9: 303 (2008)). Genes with a statistically significant difference (adjusted p-value<0.05) were tested for Gene Ontology and KEGG Pathway overrepresentation using gProfileR (v 0.6.6) software, using all genes with at least 1 read in 1 sample as background and strong hierarchical filtering (Reimand et al., Nucleic Acids Research 44, W83-W89 (2016)). The same set of genes were tested against MSigDB C2 and C5 collections for Gene Set Enrichment Analysis (GSEA) using fgsea (v 1.4.1) software with 10000 iterations (Liberzon et al., Bioinformatics 27, 1739-1740 (2011); Sergushichev, bioRxiv, (2016); Subramanian et al., Proceedings of the National Academy of Sciences 102, 15545-15550 (2005)) and the “stat” column from the DESeq2 results table as input.

### Organoids Ca²⁺2-Photon Imaging

Multiphoton imaging was performed using a custom-built multiphoton microscope (Bergamo, Thorlabs), equipped with a resonant scanner, a piezo Z drive, and a 25×1.1 NA water-immersion lens (CF175 Apochromat 25XC W, Nikon, Japan). The light source (Mai Tai eHP DeepSee, Spectra-Physics, CA) was run at 800 nm or 920 nm, and the green fluorescence was collected using a 525/50 nm bandpass filter (Chroma, VT). Cortical organoids were stained with 2 μM Fluo-4AM in the culture medium (BP Organoid Medium) for 45 minutes in 37° C. CO₂ incubator. Time-lapse images of volumetric (533×533×50 μm³ or 265×265×50 μm³) stacks were taken at 1.26 Hz for 3 min. During imaging sessions, organoids were kept at 37° C. using a plate heater (TC-324C, Warner Instruments, CT). After baseline imaging, drugs (Paxilline: 10 μM; TTX: 2 μM) were added via perfusion. 10 minutes after drug application, images were captured, analyzed and quantified using ImageJ (NIH) and MATLAB (Mathworks) as previously reported (Li et al., J Neurophysiol 98, 3341-3348 (2007); Li et al., Biophys J 98, 1733-1741 (2010)).

### Behaviors

Flurothyl-induced seizures: Experiment was conducted following the protocol adapted from previous report (Judson et al., Neuron 90, 56-69 (2016). Mice (8-9 weeks, both males and females) were put into a 1-liter transparent plastic chamber for 1 min habituation. Subsequently, chamber was capped with sealed cover and flurothyl (10% in ethanol, Sigma) was perfused onto a piece of tissue paper hung in the chamber at a rate of 100 μl/min by using a programmable syringe pump (Harvard Apparatus). The first myoclonic seizure was defined by a short but obvious jerk of a neck and body due to muscle contraction. Each trial was terminated when the mouse exhibited generalized seizure as a loss of postural control. Chamber was cleaned and the tissue paper was replaced before each new trial.

Picrotoxin-induced seizure: Mice were injected with picrotoxin (3 mg/kg, i.p., Tocris Bioscience) and placed into 1 L beakers for 30 min observation with simultaneous video recordings. Seizure severity of mice was dependent on multiple variables such as picrotoxin dose, mice age, and their body weights. We performed dosage-response experiments with a dosage from 2-5 mg/kg of picrotoxin in wildtype mice and found that 3 mg/kg of picrotoxin was optimal to induce grade 2 level seizure in wildtype mice. Videos were recorded for all the behavioural experiments. Behavioral analysis was performed in a double-blind manner with littermates (siblings of wild-type (Ube3a^{m+/p+}) and maternal knockout (Ube3a^{m-/p+}) mice that produced from same pairs of Ube3a^{m+/p-} females and Ube3a^{m+/p+} males).

### Example 2

Altered Excitability and fAHP in UBE3A-Null Human Neurons

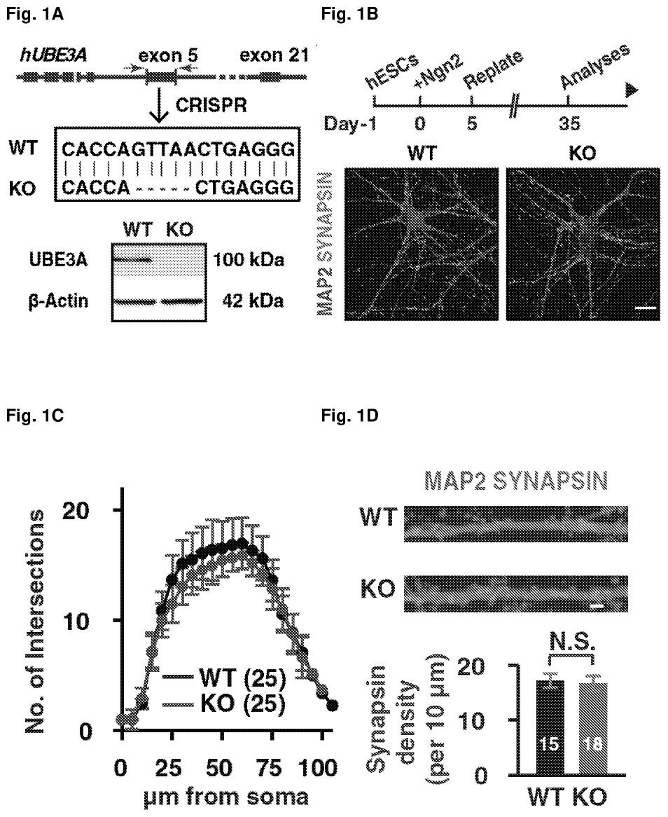

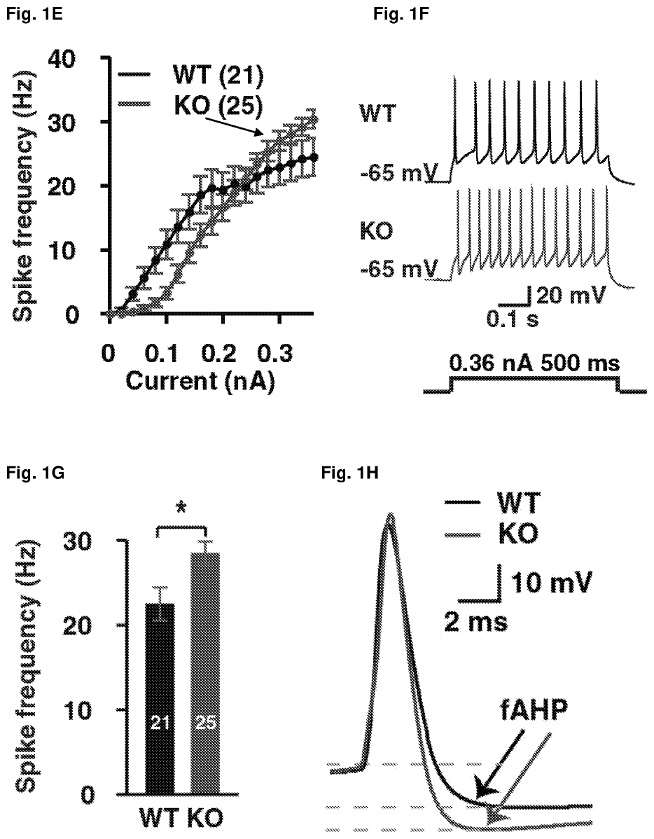

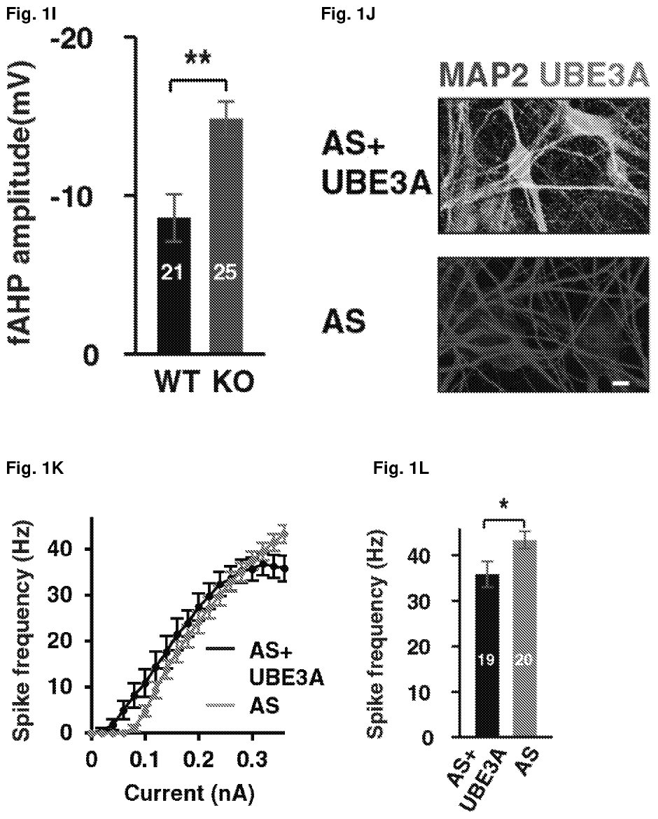

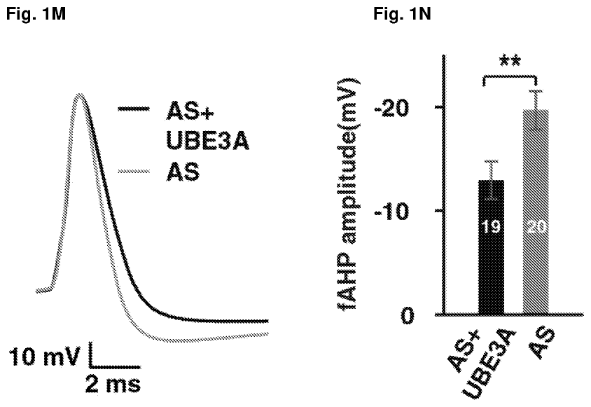

To understand the cellular and functional consequences of the loss of UBE3A in human neurons, we first generated UBE3A knockout (KO) cells in two human embryonic stem cell lines (hESC, H1 and H9) utilizing the CRISPR-Cas9 system (FIG. 1A). Sanger sequencing indicated that a resultant clone contained a 5 bp homozygous deletion in exon 6 that resulted in a frame shift and early translational termination (FIG. 1A). Western blot analysis confirmed the absence of the UBE3A protein in the KO clone (FIG. 1A). We further assessed the potential off-target effects of our CRISPR-Cas9-based knockout by sequencing the top ten predicted off-target sites of our sgRNA sequence 5′-CTACTACCACCAGTTAACTGAGG-3′ (SEQ ID NO: 20) (Table 2) and found no alteration at these sites.

TABLE 2
Top 10 off-targets.

| SEQ ID | Off-target sequence | mismatches | UCSC gene | Locus |
|---|---|---|---|---|
| 21 | CTGAGACCACTAGTTAACTGTGG | 4MMs | NM_003340 | chr4: +103715857 |
| | | [3:4:5:11] | | |
| 22 | CTCCTAAAACTAGTTAACTGTAG | 4MMs | NM_133437 | chr2: +179507114 |
| | | [3:7:8:11) | | |
| 23 | CAAATACCAGCAGTTCACTGGAG | 4MMs | NM_020762 | chr12: −64383705 |
| | | [2:4:10:16] | | |
| 24 | CTAGTCACACCAGTTACCTGAAG | 4MMs | NR_026749 | chr1: +48625310 |
| | | [4:6:7:17] | | |
| 25 | CTCCAACCCCCAGTGAACTGTGG | 4MMs | NR_036684 | chr16: −66973269 |
| | | [3:5:9:15] | | |
| 26 | CTACAACCACCAGTTTACTGCAG | 2MMs | N/A | chd7: −15850204 |
| | | [5:16] | | |
| 27 | CTGCAACCACTAGTTAACTGGAG | 3MMs | N/A | chr7: +69963608 |
| | | [3:5:11] | | |
| 28 | CTTCTACCCTCAGTTAACTGCAG | 3MMs | N/A | chr12: +101671903 |
| | | [3:9:10] | | |
| 29 | TTTCAACAACCAGTTAACTGGAG | 4MMs | N/A | chr2: +5123598 |
| | | [1:3:5:8] | | |
| 30 | CTATTACCAGCAGTTATCTGAGG | 3MMs | N/A | chr8: −77722678 |
| | | [4:10:17] | | |

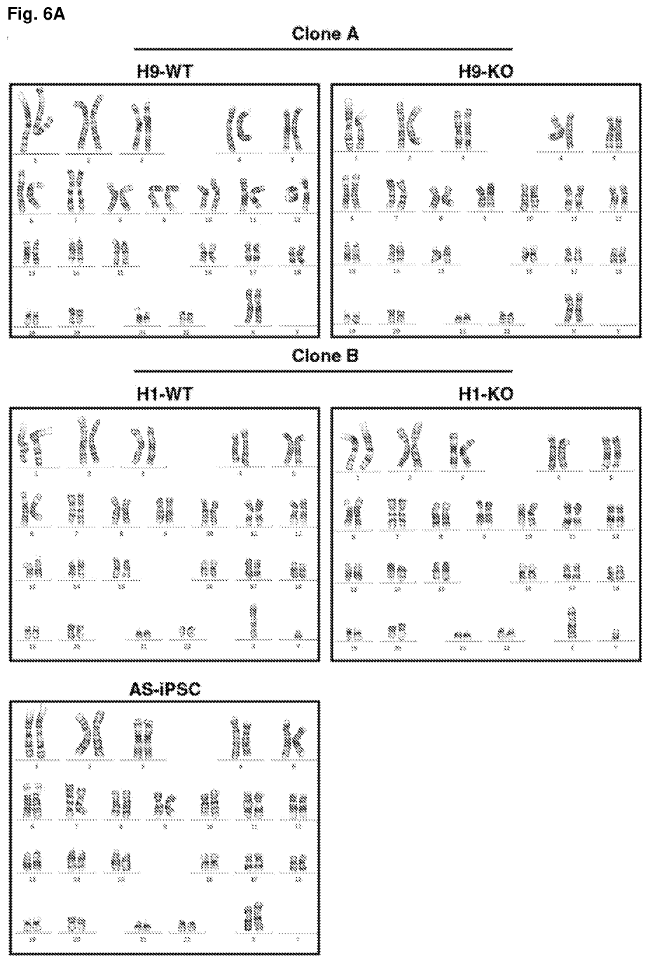

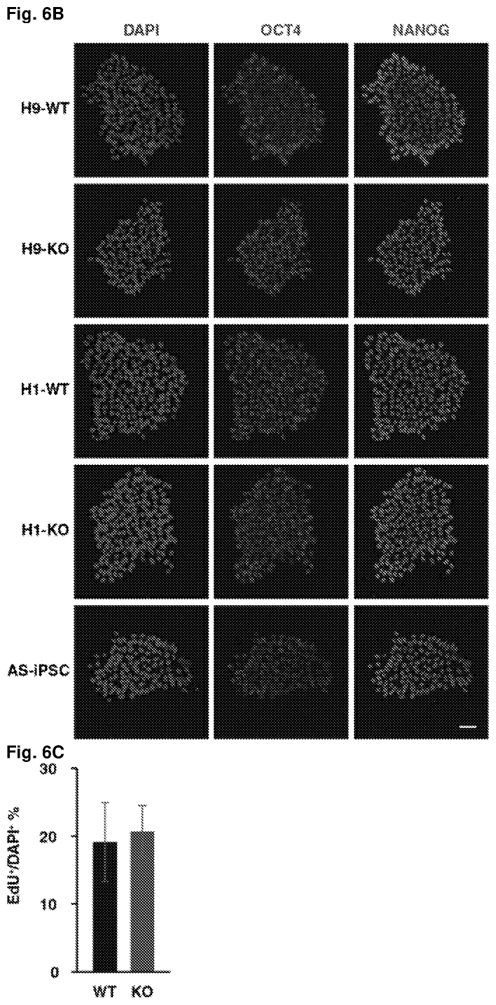

UBE3A KO hESCs were karyotypically normal (FIG. 6A), expressed high levels of key pluripotency genes and proliferated in a manner similar to that of the parental wildtype (WT) cells (FIGS. 6B and 6C).

Next, we induced the differentiation of neurons from these isogenic hESCs by ectopically expressing Ngn2 (Zhang et al., Neuron 78, 785-798 (2013)). This protocol generates homogenous populations of electrically mature cortical neurons in a time frame shorter than that achieved by morphogen-guided differentiation protocols (Yi et al., Science 352(6286): aaf2669 (2016)). Both UBE3A WT and KO hESCs were converted to neurons with similar efficiency (FIG. 7A). At 35 days post-induction, we observed morphologically mature neurons with dendritic arborizations decorated with synaptic markers (FIG. 1B). There was no significant difference in dendritic complexity or the number of morphological synapses between WT and KO neurons (FIGS. 1C and 1D). This lack of morphological alterations in KO neurons prompted us to investigate functional changes using whole-cell patch clamp recordings. By injecting stepped currents into neurons to trigger action potentials (APs), we observed that currents higher than 300 pA consistently resulted in increased AP firing frequencies in KO neurons (FIGS. 1E-1G). There were no differences in membrane capacitance, input resistance, AP threshold, and AP amplitudes (spike amp) between WT and KO neurons (FIGS. 7B-7E). We therefore monitored changes in voltage-gated sodium (V-Na⁺) and potassium (V-K⁺) channels, which are essential for AP generation (Bean, Nat Rev Neurosci 8, 451-465 (2007)). Current-voltage characteristic (I-V) curves induced by stepped depolarization were indistinguishable between WT and KO neurons (FIG. 7F). However, when we examined the fast component of afterhyperpolarizations (fAHPs) (FIG. 7G), which can affect neuronal excitability in a bidirectional manner (Gu et al., J Physiol 580, 859-882 (2007); Kimm et al., J Neurosci 35, 16404-16417 (2015); Montgomery and Meredith, Proc Natl Acad Sci USA 109, 18997-19002 (2012)), we observed that fAHPs were significantly augmented in UBE3A KO neurons (WT: −8.6±1.5 mV; KO: −14.8±1.1 mV; p=0.0014) (FIGS. 1H and 1I). The changes in AP firing and fAHP in KO neurons were restored by the ectopic expression of WT UBE3A (FIGS. 7H-7L), confirming that the lack of UBE3A was responsible for these functional changes. To verify that these differences were not specific to the particular hESC line (H9), we generated an independent pair of isogenic UBE3A WT/KO clones in another hESC line (H1) (FIG. 7M) and were able to reproduce similar functional changes (FIGS. 7N-7S). In humans, AS is associated with the loss of the maternal UBE3A allele, and this could not be recapitulated in our homozygous KO clones. Thus, we sought to determine whether neurons from AS patient-derived iPSCs exhibit a similar excitability increase with augmented fAHP. To this end, we generated neurons from an AS iPSC line carrying a microdeletion in chromosome 15q11.2-q13.1 (which includes the UBE3A gene) and confirmed that UBE3A protein was not observed in induced neurons (FIG. 1J). The introduction of WT UBE3A into these AS iPSC-derived neurons similarly rescued functional changes in AP firing and in fAHPs (FIGS. 1K-1N). Taken together, our results establish that the loss of UBE3A in human neurons resulted in altered neuronal excitability associated with enhanced fAHP.

### Example 3

BK Augmentation Underlies Increased fAHP

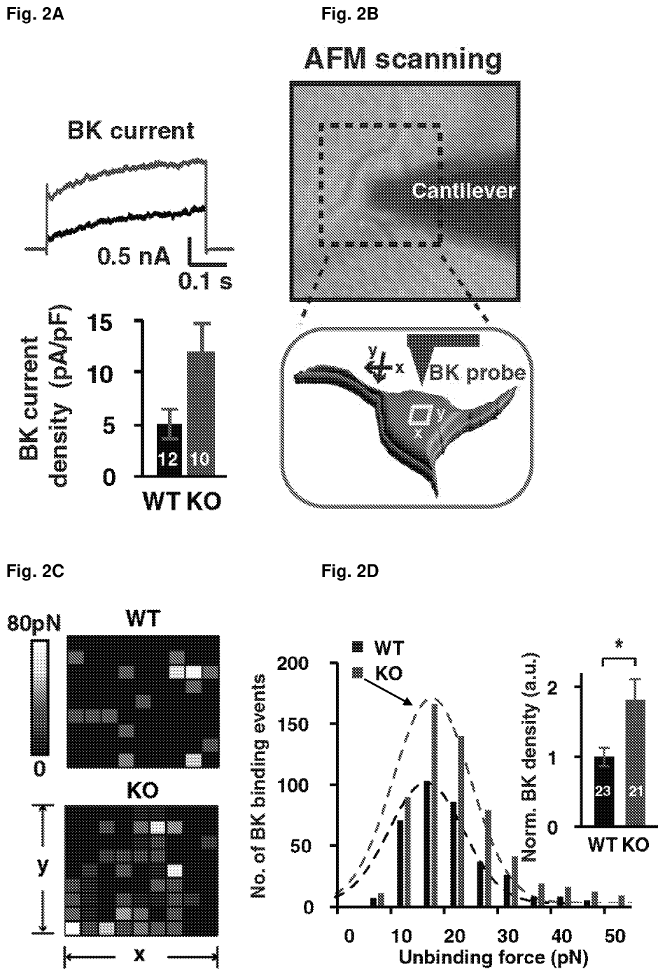

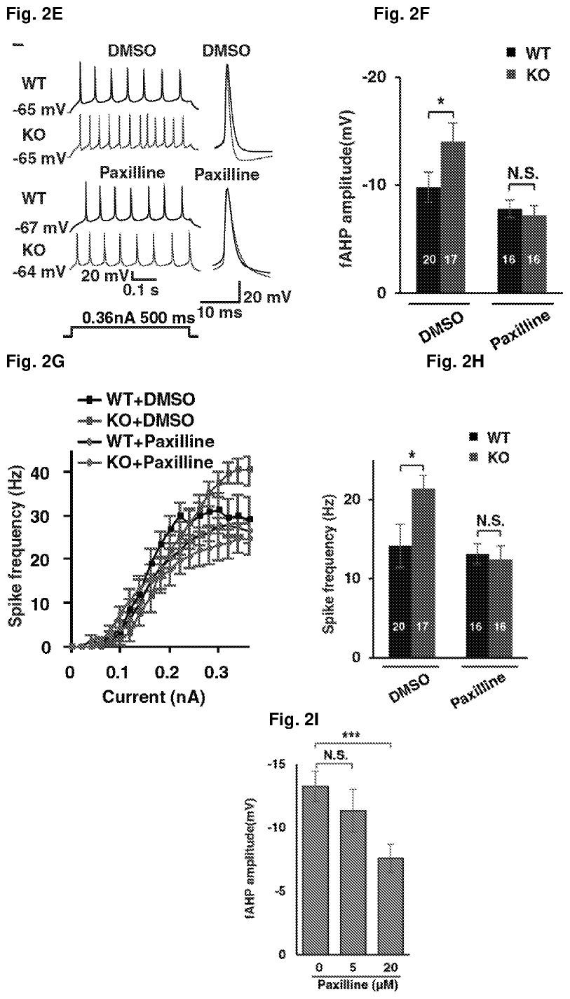

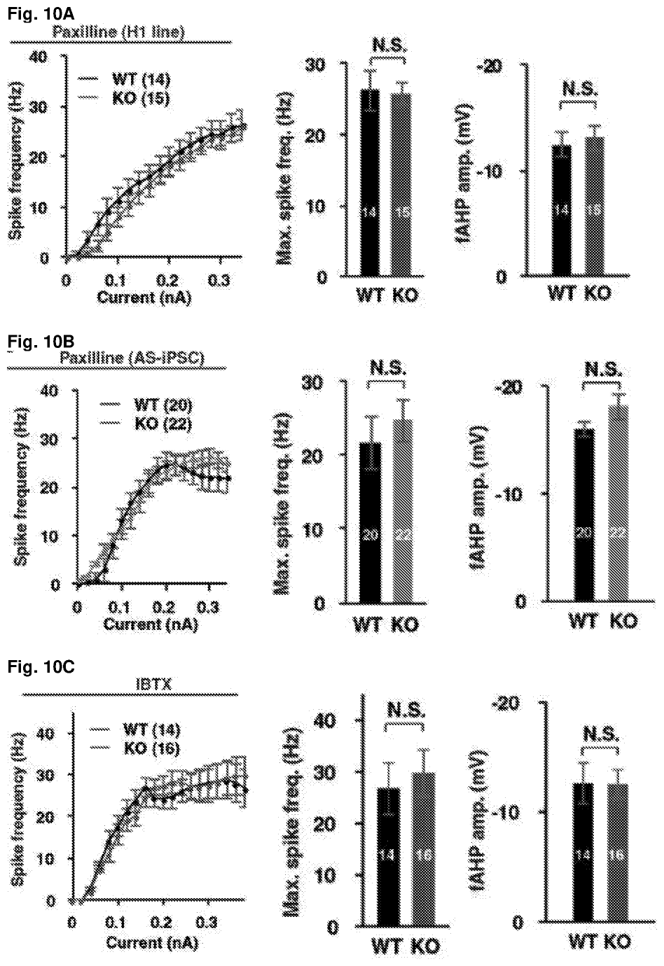

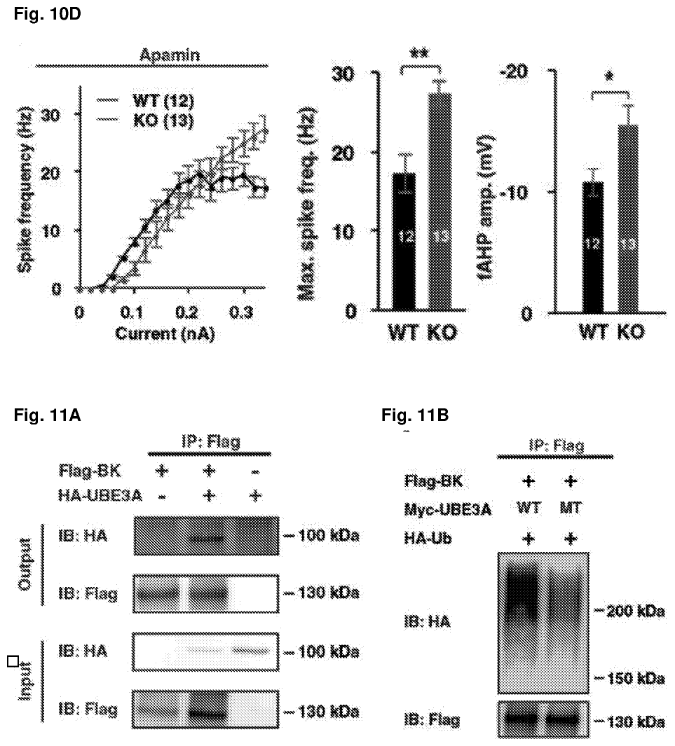

fAHPs are primarily mediated by calcium- and voltage-dependent big potassium (BK) channels in neurons (Storm, J Physiol 385, 733-759 (1987)). To determine whether the enhanced fAHPs observed in KO neurons resulted from increased BK channel activity, we pharmacologically isolated BK currents (FIG. 8A). Compared with WT-derived neurons, UBE3A KO neurons exhibited larger BK currents (FIG. 2A and FIG. 8B). Although a previous report indicated that SK2 (small-conductance-activated K⁺ channel 2) is a substrate of UBE3A (Sun et al., Cell Rep 12, 449-461 (2015)), we did not observe any significant difference in SK2-mediated medium AHPs (FIGS. 8C and 8D). The increased BK currents observed in KO neurons might simply be due to increased BK protein expression. Indeed, western blot analyses showed that the level of the obligatory subunit of BK (BKα) was higher in KO neurons (FIGS. 8E and 8F). We independently confirmed this finding by employing single-molecule, atomic force microscopy (AFM) to assess the density of BK proteins on cellular membranes by analyzing the physical interactions between BK proteins and BK-specific antibodies conjugated to the AFM tip (FIG. 2B and FIG. 9) (Maciaszek et al., J Neurosci 32, 11435-11440 (2012)). Sequential AFM force mapping was conducted by spatially moving the functionalized tip on the soma membranes of neurons. FIG. 2C shows an example of a heatmap in which the shading of each block indicates the presence of BK channels at that location. The results revealed that the BK channel density was higher on the membranes of KO cells (FIGS. 2C and 2D). If increased BK levels led to enhanced fAHP and elevated excitability in KO neurons, we hypothesized that treatment with a BK antagonist would normalize these differences. Indeed, application of BK antagonists (either 5 μM paxilline or 100 nM IBTX) (FIGS. 2E-2H and FIGS. 10A-10C), but not an SK antagonist, apamin (200 nM) (FIG. 10D), normalized the differences in fAHP amplitude and AP firing frequency between KO and WT neurons, demonstrating that BK channels mediate the augmented fAHP observed in KO neurons. Inactivation of BK channel by a relatively low dose (5 μM) did not significantly reduce the fAHP in WT neurons (FIG. 2I). Intriguingly, 5 μM of paxilline in KO neurons successfully restored excitability and fAHP to WT levels (FIG. 2E-2H), thus demonstrating that abnormal neuronal properties resulting from lack of UBE3A are sensitive to BK inactivation by paxilline. However, 20 μM paxilline treatment significantly lowered fAHP in WT, which will make neurons more excitable (FIG. 2I). Taken together, these data suggest that only certain dosage of paxilline is necessary to normalize KO neurons' functional defects.

### Example 4

### UBE3A Mediates Ubiquitination and Proteasomal Degradation of BK

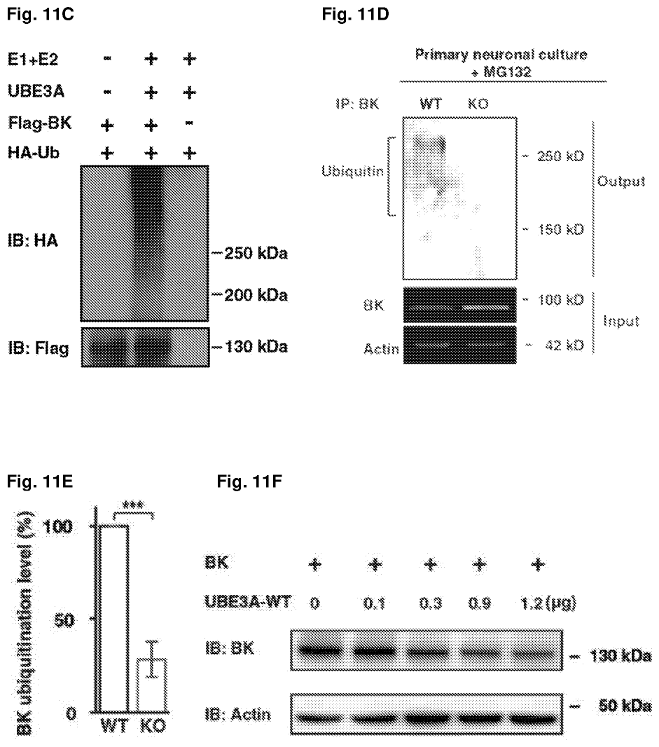

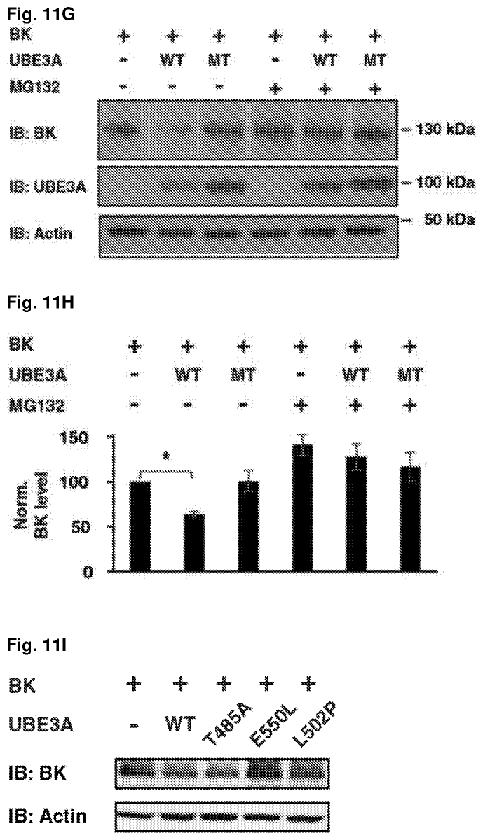

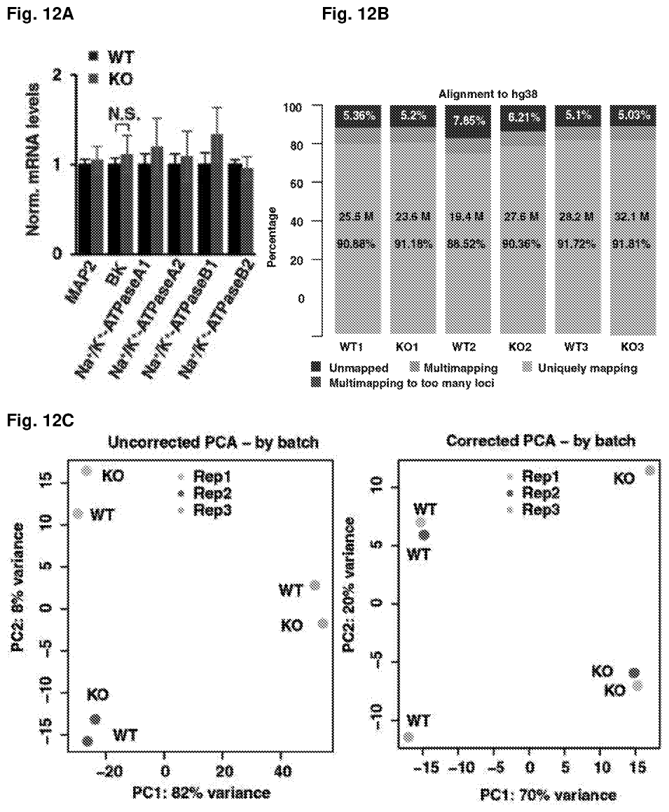

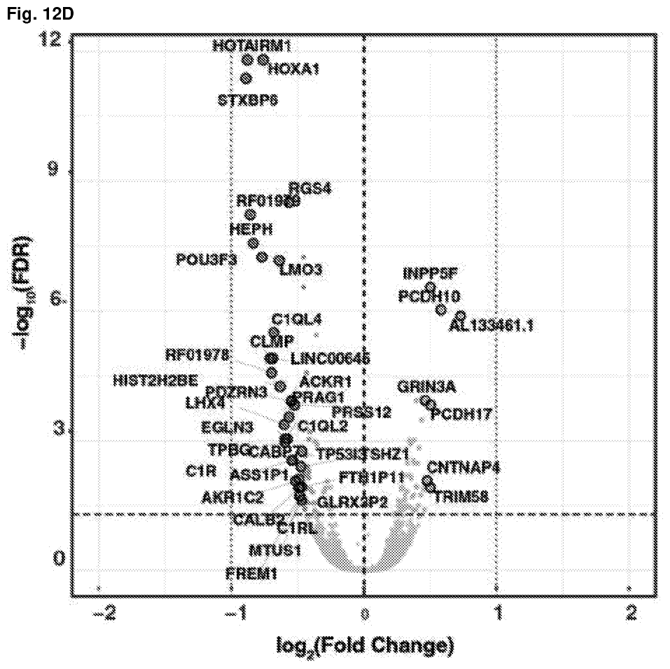

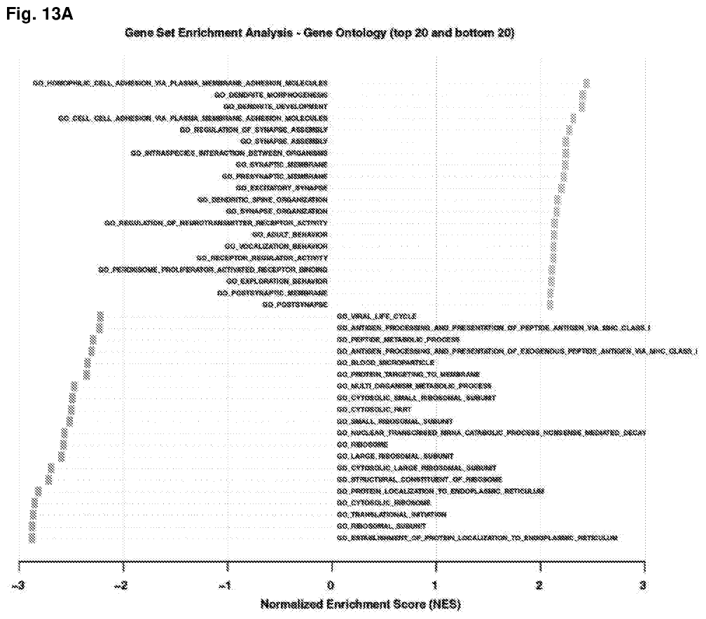

Next, we sought to determine how the loss of UBE3A led to BK augmentation. Since UBE3A is an E3 ligase, we hypothesized that BK is one of the substrates of UBE3A-mediated ubiquitination and proteasomal degradation (Sell and Margolis, Front Neurosci 9, 322 (2015)). To test this hypothesis, we first performed in vitro coimmunoprecipitation (IP) in heterologous cells and confirmed that there is an interaction between UBE3A and BK (FIG. 11A). Second, when FLAG-tagged BK was coexpressed with hemagglutinin (HA)-tagged ubiquitin, we observed more BK ubiquitination when WT UBE3A was present than when a catalytic-dead mutant (MT, C885A) was used (FIG. 11B). BK ubiquitination by UBE3A was further confirmed by an in vitro ubiquitination assay using recombinant proteins (FIG. 11C) and by assessing endogenous BK ubiquitination using mouse cortical neurons derived from either WT or Ube3a^{m-/p+} mice (FIGS. 11D-11E). Furthermore, coexpressing BK cDNA with increasing amounts of UBE3A cDNA resulted in a corresponding decline in the BK protein level, and this decrease in the BK level was abolished by the proteasome inhibitor MG312 (FIGS. 11F-11H). Collectively, these data demonstrate that BK is one of the targeted substrates of UBE3A-mediated ubiquitination and proteasomal degradation. A small subset of AS cases do not carry genetic deletions in UBE3A but instead possess missense mutations in UBE3A (Mabb et al., Trends Neurosci 34, 293-303 (2011)). Ectopically expressing AS-associated UBE3A missense mutants (E550L, L502P) (Sadikovic et al., Hum Mutat 35, 1407-1417 (2014)) failed to downregulate BK protein levels. However, downregulation of BK protein level was observed when an autism-associated mutant (T485A) was ectopically expressed (Yi et al., J Biol Chem 292, 12503-12515 (2017)), (FIG. 11I), indicating that increased neuronal BK levels may also be a pathophysiological phenotype in AS patients with missense mutations in UBE3A. Although our data thus far suggest that UBE3A affects BK protein abundance via its E3 ligase activity, UBE3A could regulate BK levels via transcriptional modulation (Ramamoorthy and Nawaz, Nucl Recept Signal 6, e006 (2008)). However, quantitative real-time RT-PCR revealed that there was no significant difference in BKα subunit expression between WT and KO neurons (FIG. 12A). To identify which transcripts are altered by UBE3A deficiency at the genome-wide level, we performed mRNA sequencing on WT and KO neurons (FIGS. 12B-12D).

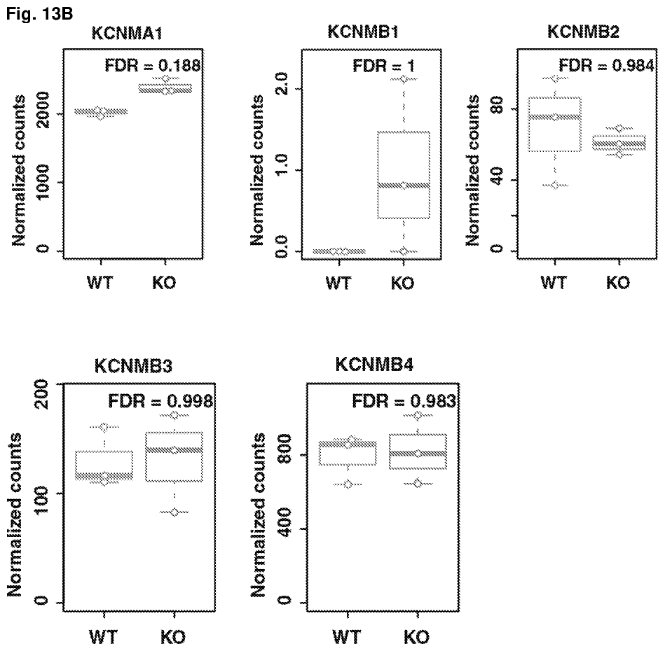

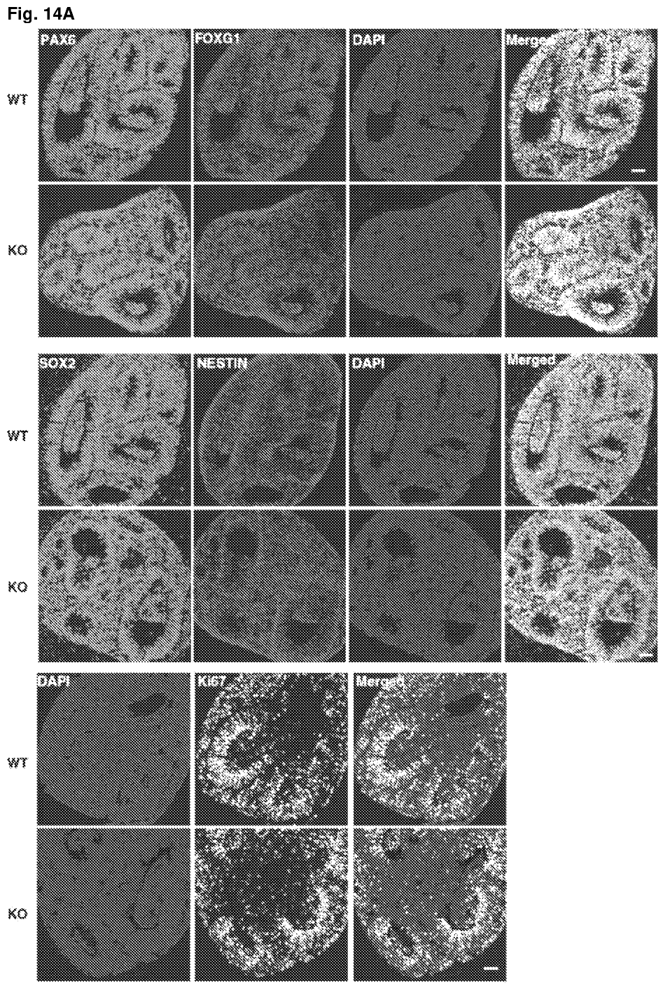

Differential expression analysis showed a slight perturbation in the transcriptome overall: in total, 129 differentially expressed genes (adjusted p<0.05, FIGS. 12D, 13A) were identified, and all of these genes showed minor changes, as indicated by the small range of log 2 (fold-change) values. Consistent with our qPCR results, we found no significant difference in the expression levels of BK channel genes (FIG. 13B). Taken together, our results support the notion that, in human neurons, UBE3A primarily regulates BK levels via a post-translational mechanism.

### Example 5

### BK Antagonist Restores Neuronal Excitability and Network Activity in 3D Organoids

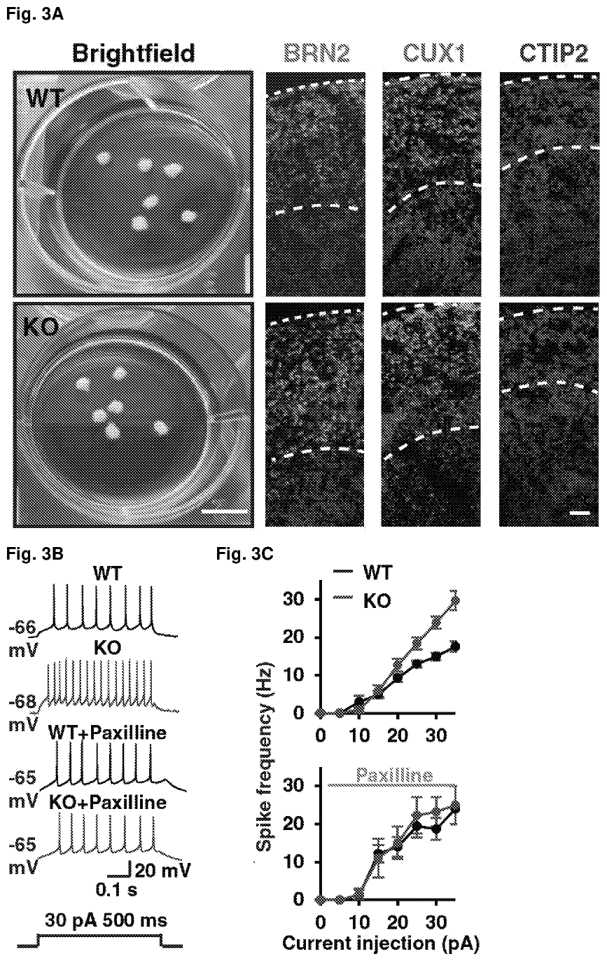

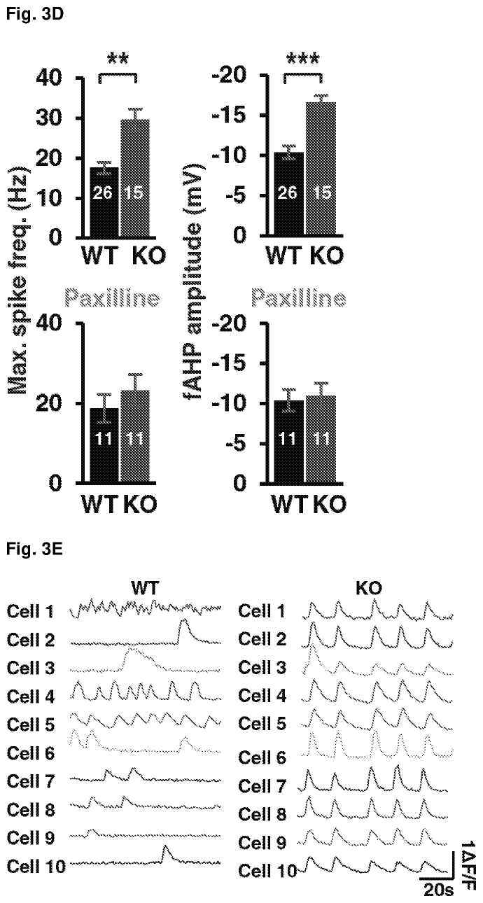

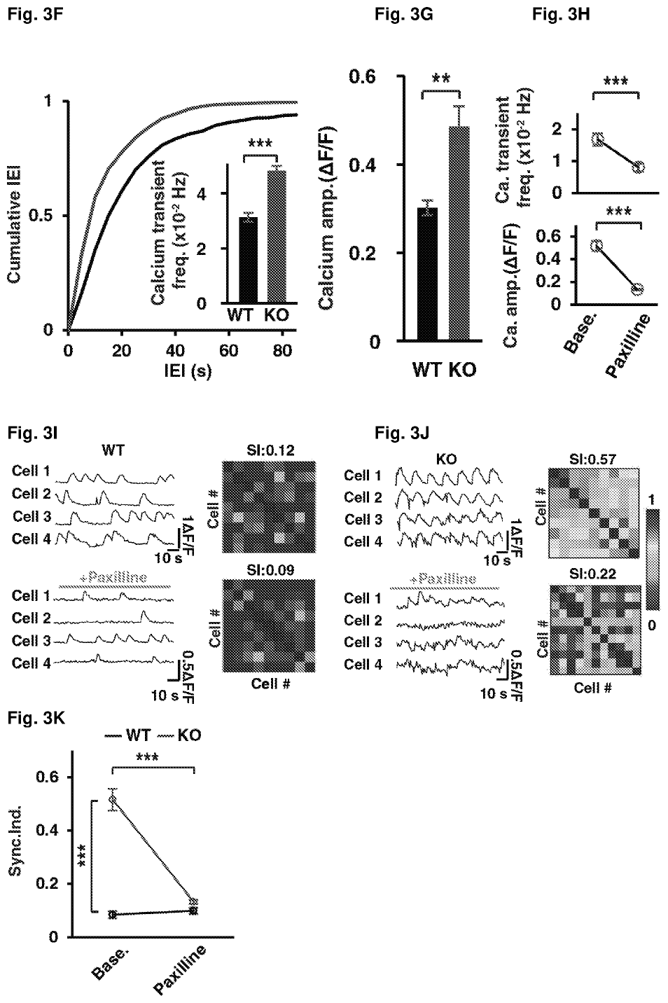

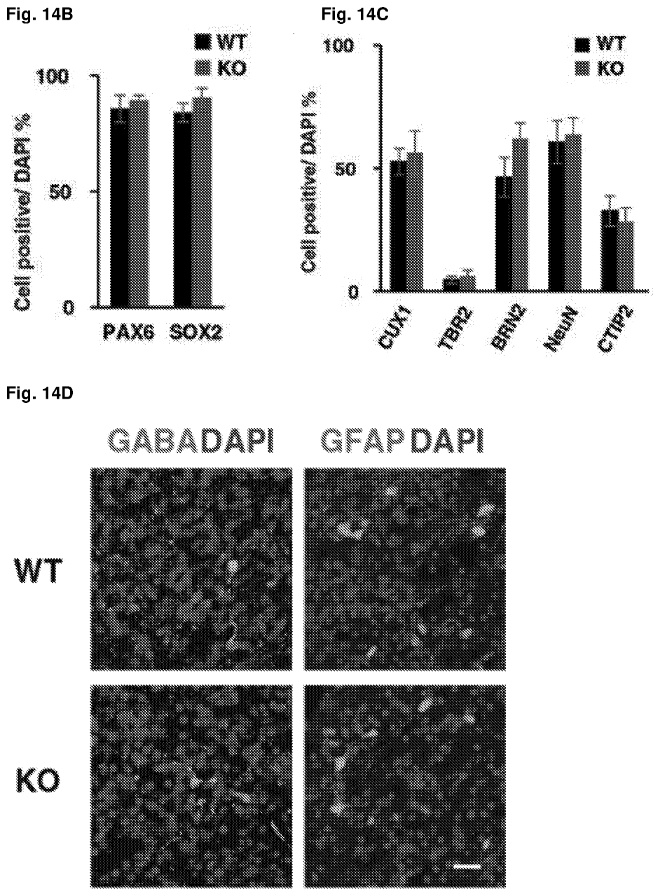

One advantage of using our single-step neuronal induction protocol instead of morphogen-guided differentiation protocols is that our protocol can produce electrically mature neurons (Zhang et al., Neuron 78, 785-798 (2013)). However, neurons obtained using this protocol do not follow a normal developmental trajectory in that they bypass the progenitor stage, raising the question of whether these functional deficits are relevant to AS disease progression. To address this issue, we generated three-dimensional (3D) cortical organoids and utilized this system to investigate functional changes in UBE3A-deficient neurons that follow a normal developmental maturation sequence (Pasca et al. Nat Methods 12, 671-678 (2015)). These brain organoids have been shown to better recapitulate the developmental time-lines of human brains (Kelava and Lancaster, Cell Stem Cell 18, 736-748 (2016)). After 20 days of differentiation (D20), both WT and KO cells produced spheroids that were indistinguishable in size. They were similarly composed of polarized, proliferating neuroepithelial cells that expressed canonical neuronal progenitor markers, including PAX6, SOX2, and NESTIN (FIGS. 14A and 14B). These brain organoids continued to grow over the next 100 days to about 1-2 mm (FIG. 3A). After 120 days, immunostaining revealed the presence of a substantial number of NeuN-positive (*) neurons. Consistent with previous reports, multiple cortical layer markers were also observed, including BRN2/CTIP2/CUX1, and these segregated the neurons into upper and deep layers (FIG. 3A). Quantification analyses showed that there was no significant difference in the number of NeuN⁺ neurons or the expression of the cortical layer markers between WT and KO organoids (FIG. 14C). We detected a few GABA+ neurons, indicating the presence of inhibitory neurons, within the organoids. GFAP+ cells, which are indicative of mature astrocytes, were also observed in both WT and KO organoids (FIG. 14D). Collectively, these data demonstrate that both the WT and KO organoids contained similar compositions of mature cortical cell types when grown in long-term cultures.

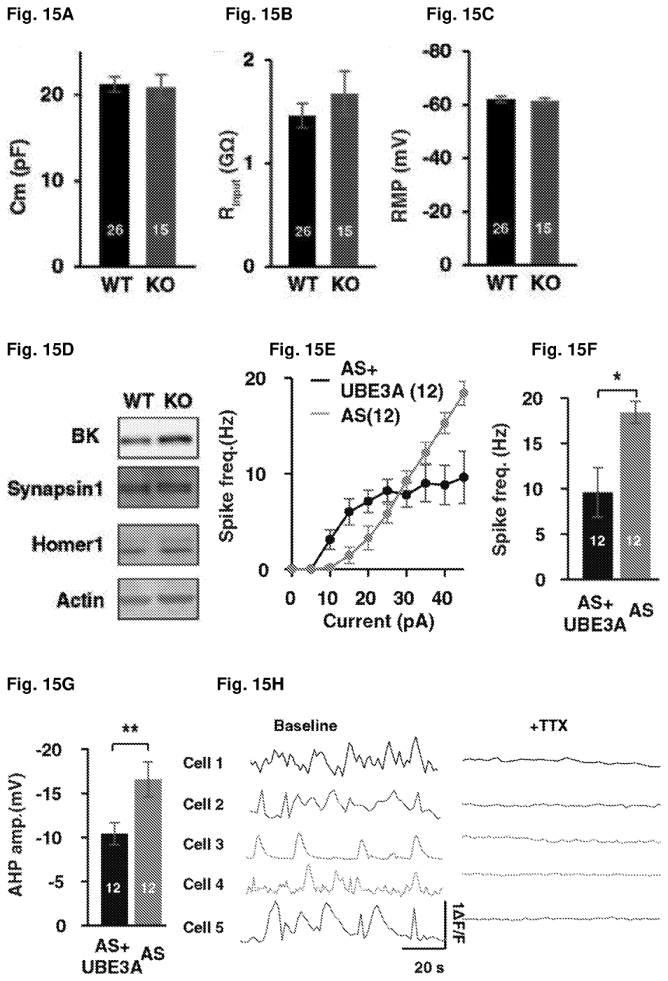

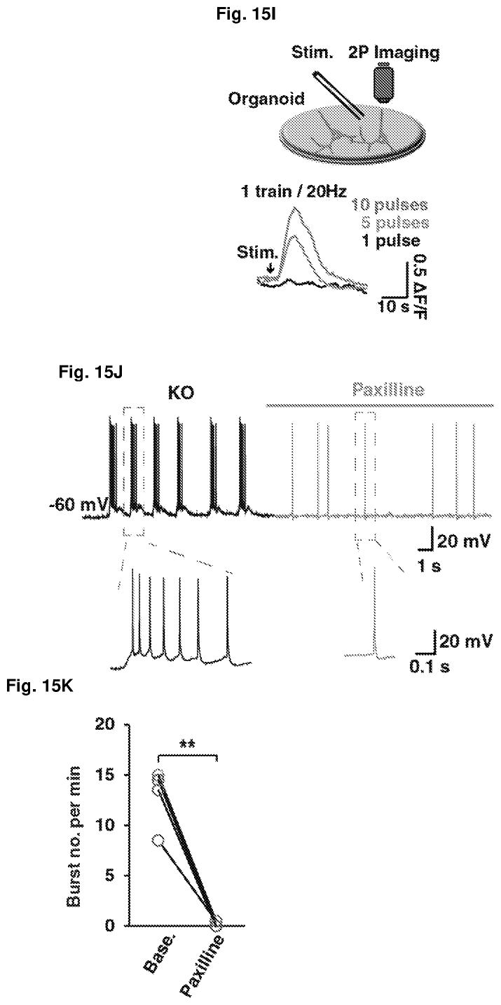

Next, we performed whole-cell patch-clamp recordings to evaluate the intrinsic properties of neurons in WT and KO organoids after 120-150 days in culture. Similar to the results obtained in 2D-induced neurons, pyramidal-shaped neurons within UBE3A KO organoids exhibited augmented excitability and elevated fAHP (FIGS. 3B-3D) while other electrophysiological properties remained the same, indicating that the overall developmental maturation is similar between WT and KO organoids (FIGS. 15A-15C). Immunoblots of BK proteins confirmed that BK levels were higher in KO organoids (FIG. 15D). Paxilline treatment normalized the augmented neuronal excitability and fAHP changes observed in neurons of KO organoids (FIGS. 3B-3D). We obtained similar results from cortical organoids generated from AS iPSCs (FIGS. 15E-15G). Thus, our recordings performed in long-term cultured 3D human cortical organoids reproduced the phenotypes observed in 2D human neurons.

Because 3D organoids are capable of assembling more sophisticated neuronal networks than that which can be achieved in 2D neuronal cultures, we sought to use calcium (Ca²⁺) imaging to monitor differences in neuronal network dynamics in large populations of cells in WT and KO cortical organoids. Incubating cortical organoids with Fluo-4AM (2 μM) resulted in labelled cells located up to 170 μm from the surface. In WT organoids, bath application of TTX (2 μM) abolished spontaneous calcium transients (FIG. 15H). While Ca²⁺ neurons in day 120-150 WT organoids displayed prominent spontaneous transients, at the network level, synchronization of transients was rare, and most neurons fired in a stochastic manner (FIG. 3E) (Kirwan et al., Development 142, 3178-3187 (2015)). In contrast, neurons in UBE3A KO organoids exhibited more frequent firing with augmented dynamic ranges of Fluo-4 fluorescence, and neurons in some KO organoids seemed to discharge regularly and synchronously (FIGS. 3E-3G). Soma calcium transients were correlated with intrinsic neuronal activity, as indicated by the finding that the intensity of somatic Fluo-4 fluorescence gradually increased along with graded electrical train stimulations in individual neurons (FIG. 15I), demonstrating that regular fluorescence fluctuations were related to intrinsic neuronal bursting, as shown in previous reports (Kerr et al., Proc Natl Acad Sci USA 102, 14063-14068 (2005); Emiliani et al., J Neurosci 35, 13917-13926 (2015)). In addition, perforated patch recordings revealed that neurons exhibit spontaneous burst-firing in UBE3A KO organoids (FIG. 15J). Paxilline treatment completely converted spontaneous burst firing into single AP firing in these neurons (FIGS. 15J and 15K) and decreased both the frequencies and the amplitudes of calcium transients in the organoids (FIG. 3H), suggesting that augmented BK activity promoted burst firing in individual neurons and subsequent hyperactivity in the entire network. Finally, we investigated network-level synchronization in cortical organoids. Indeed, we found that KO organoids and AS iPSC-derived organoids exhibited higher levels of synchronization and that this effect was abolished by treatment with paxilline (FIGS. 3I-3K). Taken together, our data suggest that aberrant intrinsic firing of UBE3A-null neurons collectively elicits network-level hyperactivity and synchronization resembling the seizure activity observed in AS patients (Pelc et al., Seizure 17, 211-217 (2008)).

### Example 6

### BK Antagonist Ameliorates Epileptic Susceptibility in AS Mouse.

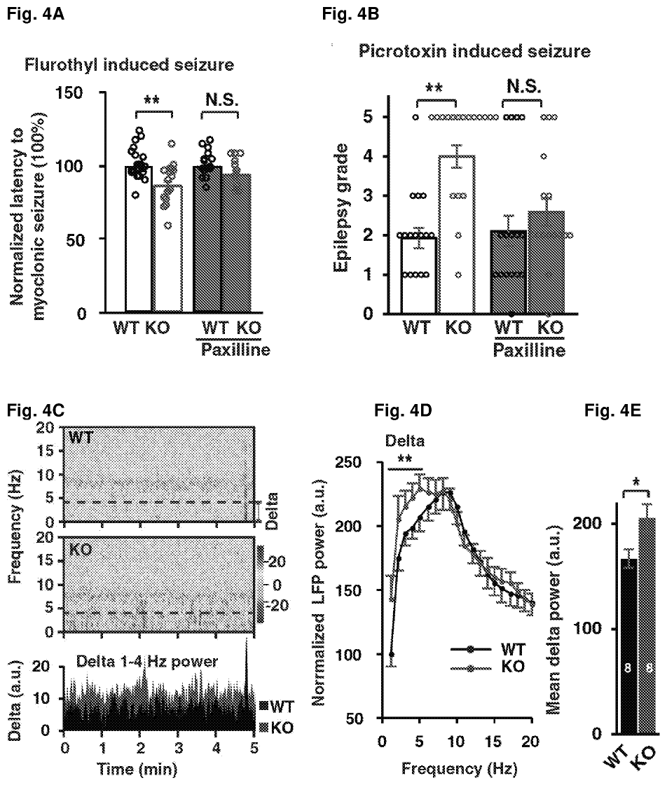

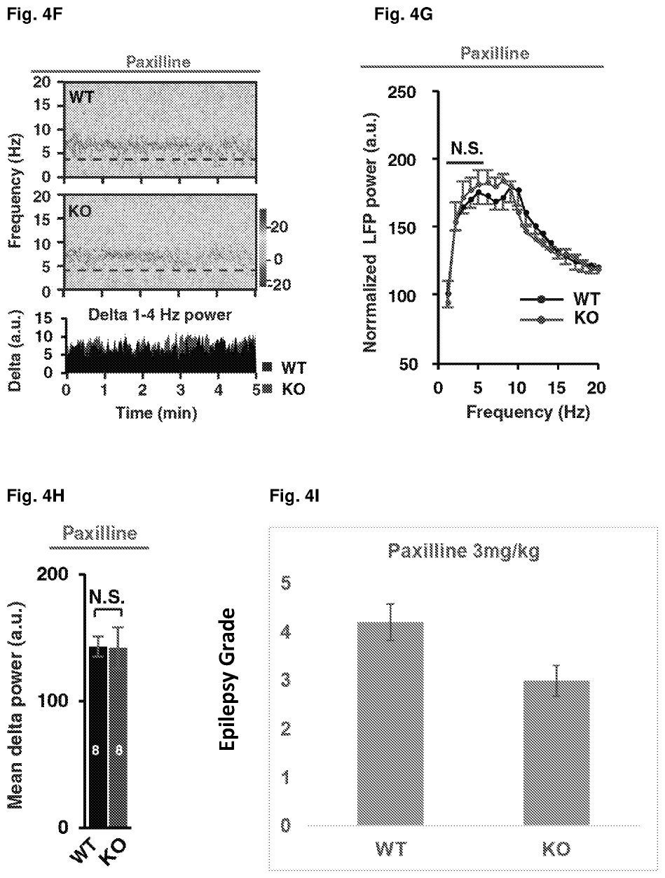

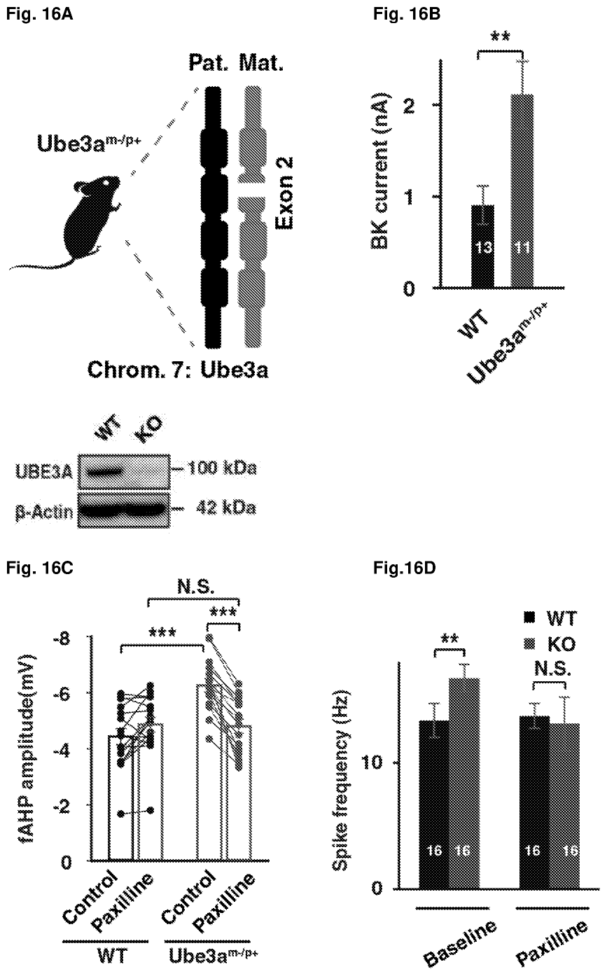

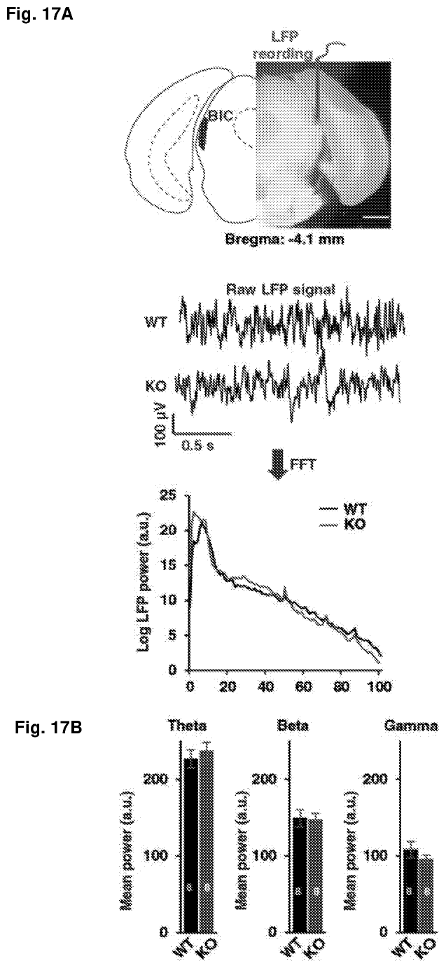

Our data thus far demonstrate that changes in intrinsic excitability are mediated by augmented BK channel activities in neurons derived from both isogenic UBE3A KO hESCs and AS iPSCs. Because none of these phenotypes had been previously reported in AS mouse models, we sought to determine whether they would also be present in neurons obtained from mice with a maternal Ube3a deletion (Ube3^{m-/p+}) (FIG. 16A) that has been shown to recapitulate many of the symptoms associated with AS in humans (Jiang et al., Curr Opin Genet Dev 8, 334-342 (1998)). At postnatal day 28, the BK currents were significantly larger in hippocampal neurons obtained from Ube3a^{m-/p+} mice than in those obtained from WT mice (FIG. 16B). Similar to human neurons, changes in excitability and comparatively higher fAHP were observed in neurons obtained from Ube3a^{m-/p+} mice (FIGS. 16C and 16D) (fAHP; WT: −4.4±1.4 mV, n=26; Ube3a^{m-/p+}: −6.4±1.2 mV, n=29; p<0.001). Paxilline normalized these changes (FIGS. 16C and 16D). These results indicate the presence of evolutionally conserved BK augmentation in mouse neurons that lack Ube3a, and prompted us to test whether enhanced seizure susceptibility observed in Ube3am−/p+ mice (Judson et al., Neuron 90, 56-69 (2016)) could be normalized upon BK antagonist treatment. First, we exposed mice to the putative GABA_{A}R antagonist, flurothyl (100 μl/min) and observed that Ube3^{am-/p+} mice exhibit significant reduction in the latency to myoclonic seizure, and this effect could be normalized by paxilline pre-treatment (0.35 mg/kg, intraperitoneal (i.p.) injection) (FIG. 4A). Flurothyl treatment often triggered behavioral responses too quickly to grade the epileptic level, therefore, we turned to picrotoxin, another antagonist of GABA_{A} receptor, to induce seizures. Similarly, picrotoxin (3 mg/kg, i.p.), induced grade 1-2 seizure based on Racine's scale (Racine, Electroencephalogr Clin Neurophysiol 32, 281-294 (1972)) in WT mice, while the same treatment induced a much more severe seizure phenotype (grade 5) in Ube3a^{m-/p+} mice (FIG. 4B). Paxilline pre-treatment (0.35 mg/kg, i.p.) was able to normalize the seizure grade of Ube3a^{m-/p+} mice to a level comparable to that observed in WT mice (FIG. 4B). Next, to investigate the AS-like electroencephalogram (EEG) abnormalities observed in these mice, we recorded resting-state local field potentials (LFPs), which are analogous to intracortical EEG, in freely moving mice (FIG. 17A) (Sidorov et al., J Neurodev Disord 9, 17 (2017)). As previously reported, compared to the WT mice, Ube3a^{m-/p+} mice exhibited a strong trend toward total spectral power being increased in the delta rhythmicity (1-4 Hz) (FIG. 4, C-E, p=0.0044) (Sidorov et al., J Neurodev Disord 9, 17 (2017)). We also observed increased power in other bands, including theta (4-8 Hz), beta (12-30 Hz) and gamma (30-80 Hz) bands, in Ube3a^{m-/p+} mice, but these differences were not statistically significant (FIG. 17B). Because increased delta rhythmicity has been linked to seizure susceptibility of Ube3a^{m-/p+} mice (Judson et al., Neuron 90, 56-69 (2016)), we sought to determine whether paxilline treatment could normalize the delta rhythmicity. Indeed, paxilline treatment normalized the enhancement observed in the delta band in Ube3a^{m-/p+} mice (p=0.85) (FIG. 4, F-H).

Typically, outward current through potassium channels during the AHP hyperpolarizes and stabilizes the membrane potentials, and complete blockade of BK channel function could result in decreased fAHP. However, in UBE3A KO neurons, BK protein is degraded less and this results in enhanced BK channel function and a larger fAHP. The fAHP can mediate either excitation or inhibition, depending on the exact membrane potential and how rapidly the BK conductance is activating or recovering. In support of this, both loss-of-function and gain-of-function mutations of the BK channel can trigger increased neuronal excitability.

Interestingly, a high dose of paxilline (3 mg/kg, compared to 0.35 mg/kg in FIG. 4B) resulted in increased seizure susceptibility in WT mice similar to KO mice without paxilline treatment (cf. FIG. 4I and FIG. 4B). While KO animals still responded positively to a high dose of paxilline, albeit without much difference in the seizure susceptibility between the two doses, the marked effect high dose paxilline had on WT indicates there is a critical dosage window for safe use of paxilline in WT. The reduced seizures in the KO mice with both concentrations of paxilline (0.35 mg/kg and 3 mg/kg) may be due to augmented BK channels in neurons as default, although still higher doses of paxilline may be expected to eventually block BK channel activity. We have some data indicating 10 mg/kg paxilline caused seizures in WT and KO mice. Taken together, we conclude that BK augmentation similar to that observed in human UBE3A KO neurons was observed in neurons of Ube3a^{m-/p+} mice and paxilline treatment ameliorated the seizure threshold as well as the high delta oscillation observed in these mice.

### Example 7

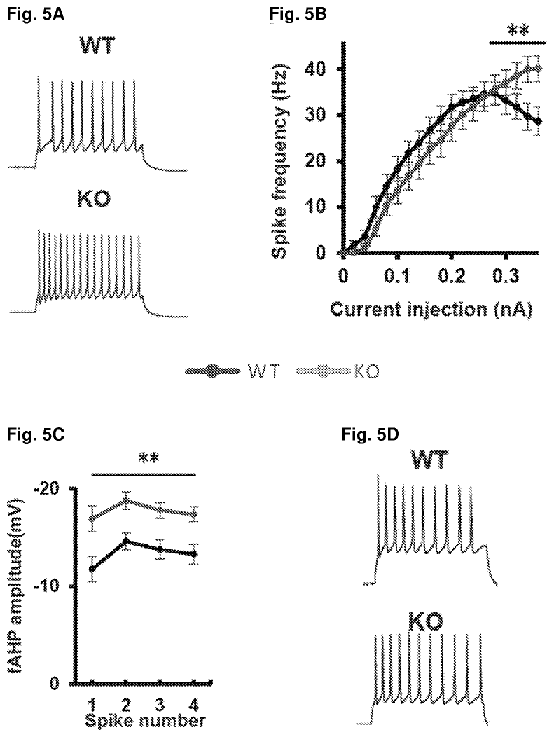

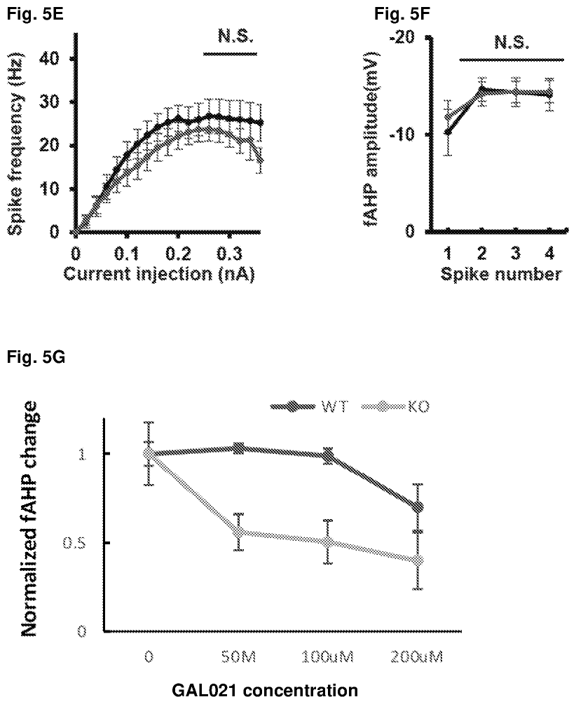

We further tested whether a new class of BK antagonist (GAL-021), which is being tested in clinical trials for potential uses in post-operative care, would normalize enhanced fAHP and elevated excitability in KO human neurons. Briefly, iPSCs were dissociated with TrypLE Express to single cells and plated onto Matrigel-coated cell culture plates in mTeSR1 media supplemented with thiazovivin (RhoA inhibitor, 1 μM). Next day, cells were transduced with lentiviral particles expressing Ngn2. At day 5, cells were dissociated again to single cells by TrypLE express and re-plated onto Matrigel-coated glass coverslips for electrophysiological recordings in BrainPHys media supplemented with SM1 and antibiotic-antimycotic drug. Primary glial cells were added onto human induced neuronal cultures at day 7. Neurotrophic factors (BDNF, GDNF, NT-3, each at 10 ng/ml) and 1% FBS and FuDR were added starting at day 10 and electrophysiological recordings were performed after day 21 to test the effects of three different dosages of GAL-021 (50 μM, 100 μM, and 200 μM) on fAHP and AP firing. FIGS. 5A-5G show that GAL-021 application returns to normal the intrinsic excitability associated with augmented fAHP in UBE3A-deficient human neurons.

It was observed that the application of GAL-021 (50 or 100 μM) normalized the differences in fAHP amplitude and AP firing frequency between KO and WT neurons (FIGS. 5A-5F). We then applied a higher concentration (200 μM) of GAL-021 in either WT or KO neurons and measured the change in fAHP amplitude and AP firing frequencies.

Interestingly, we observed that the application of GAL-021 (50 or 100 μM) did not change fAHP amplitude and AP firing frequencies, but 200 μM GAL021 resulted in decreased fAHP in the WT neurons possibly due to near complete blockade of BK channel function in these WT neurons. In contrast, 200 μM GAL021 resulted in normalized fAHP in KO neurons. These data indicate the dual-action or bidirectional nature of BK modulation, fAHP, and neuronal excitability and there are broader dosage windows amenable to modulate BK channel activities in KO neurons, compared to WT neurons.

### Summary

The inventors generated homogenous populations of electrically mature human induced cortical neurons derived from both AS patient derived iPSCs (with microdeletion of UBE3A) and isogenic UBE3A knockout cells created via CRISPR-Cas9 genome editing. Using these cells, specific changes in the intrinsic excitability of UBE3A deficient neurons were observed. These changes were caused by an increase in the fast-component of afterhyperpolarization (fAHP), which is mediated by big conductance calcium-activated potassium (BK) channels.

The application of BK channel antagonists normalized changes in neuronal excitability, fAHP, and network hyperactivity and synchronization in human induced neurons from UBE3A knockout stem cells.

Mechanistically, multiple assays, including atomic force microscopy (AFM), electrophysiology, and biochemistry, were used to demonstrate UBE3A-mediated BK ubiquitination and proteasomal degradation.

Using a more physiological model, 3D human cortical organoids were able to reproduce the intrinsic excitability changes and augmented fAHP by increasing BK activity in the UBE3A-deficient cortical organoids. Moreover, by using multi-photon Ca2+ imaging to monitor organoid-wide neuronal activities, UBE3A-lacking neurons were shown to exhibit spontaneous burst firings, which consequently induced remarkable network hyperactivity and synchronization.

The application of BK channel antagonists (paxilline, IBTX and GAL-021) normalized changes in neuronal excitability, fAHP, and network hyperactivity and synchronization in human cortical organoids derived from UBE3A knockout stem cells.

Results suggest there is a dose range window within which the BK channel antagonists were effective.

Any listing or discussion of an apparently prior-published document in this specification should not necessarily be taken as an acknowledgement that such document is part of the state of the art or is common general knowledge.

### REFERENCES

1. B. P. Bean, The action potential in mammalian central neurons. Nat Rev Neurosci 8: 451-465 (2007).
2. K. Buiting, C. Williams, B. Horsthemke, Angelman syndrome—insights into a rare neurogenetic disorder. Nat Rev Neurol 12: 584-593 (2016).
3. S. J. Chamberlain et al., Induced pluripotent stem cell models of the genomic imprinting disorders Angelman and Prader-Willi syndromes. Proc Natl Acad Sci USA 107: 17668-17673 (2010).
4. T. Conway et al., Xenome—a tool for classifying reads from xenograft samples. Bioinformatics 28: i172-178 (2012).
5. A. Dobin et al., STAR: ultrafast universal RNA-seq aligner. Bioinformatics 29: 15-21 (2013).
6. V. Emiliani, A. E. Cohen, K. Deisseroth, M. Hausser, All-Optical Interrogation of Neural Circuits. J Neurosci 35: 13917-13926 (2015).
7. J. J. Fink et al., Disrupted neuronal maturation in Angelman syndrome-derived induced pluripotent stem cells. Nat Commun 8: 15038 (2017).
8. N. Gu, K. Vervaeke, J. F. Storm, BK potassium channels facilitate high-frequency firing and cause early spike frequency adaptation in rat CA1 hippocampal pyramidal cells. J Physiol 580: 859-882 (2007).
9. Y. H. Jiang et al., Mutation of the Angelman ubiquitin ligase in mice causes increased cytoplasmic p53 and deficits of contextual learning and long-term potentiation. Neuron 21: 799-811 (1998a).
10. Y. Jiang, T. F. Tsai, J. Bressler, A. L. Beaudet, Imprinting in Angelman and Prader-Willi syndromes. Curr Opin Genet Dev 8: 334-342 (1998b).
11. M. C. Judson et al., GABAergic Neuron-Specific Loss of Ube3a Causes Angelman Syndrome-Like EEG Abnormalities and Enhances Seizure Susceptibility. Neuron 90: 56-69 (2016).
12. M. C. Judson et al., Decreased Axon Caliber Underlies Loss of Fiber Tract Integrity, Disproportional Reductions in White Matter Volume, and Microcephaly in Angelman Syndrome Model Mice. J Neurosci 37: 7347-7361 (2017).
13. I. Kelava, M. A. Lancaster, Stem Cell Models of Human Brain Development. Cell Stem Cell 18: 736-748 (2016).
14. J. N. Kerr, D. Greenberg, F. Helmchen, Imaging input and output of neocortical networks in vivo. Proc Natl Acad Sci USA 102: 14063-14068 (2005).
15. T. Kimm, Z. M. Khaliq, B. P. Bean, Differential Regulation of Action Potential Shape and Burst-Frequency Firing by BK and Kv2 Channels in Substantia Nigra Dopaminergic Neurons. J Neurosci 35: 16404-16417 (2015).
16. P. Kirwan et al., Development and function of human cerebral cortex neural networks from pluripotent stem cells in vitro. Development 142: 3178-3187 (2015).
17. A. Li et al., Molecular mechanistic insights into the endothelial receptor mediated cytoadherence of Plasmodium falciparum-infected erythrocytes. PLoS One 6: e16929 (2011).
18. Y. Liao, G. K. Smyth, W. Shi, The Subread aligner: fast, accurate and scalable read mapping by seed-and-vote. Nucleic Acids Res 41: e108 (2013).
18. J. T. Leek, J. D. Storey, A general framework for multiple testing dependence. Proc Natl Acad Sci USA 105: 18718-18723 (2008).
19. J. T. Leek, W. E. Johnson, H. S. Parker, A. E. Jaffe, J. D. Storey, The sva package for removing batch effects and other unwanted variation in high-throughput experiments. Bioinformatics 28: 882-883 (2012).
20. Leek J T, Johnson W E, Parker H S, Fertig E J, Jaffe A E, Storey J D, Zhang Y, Torres L C (2019). sva: Surrogate Variable Analysis. R package version 3.32.1.
21. Li, X., Cui, D., Jiruska, P., Fox, J. E., Yao, X., and Jefferys, J. G. Synchronization measurement of multiple neuronal populations. J Neurophysiol 98: 3341-3348 (2007).
22. Li, X., Ouyang, G., Usami, A., Ikegaya, Y., and Sik, A. Scale-free topology of the CA3 hippocampal network: a novel method to analyze functional neuronal assemblies. Biophys J 98: 1733-1741 (2010).
23. A. Liberzon et al., Molecular signatures database (MSigDB) 3.0. Bioinformatics 27: 1739-1740 (2011).
24. Y. B. Lim, J. Thingna, J. Cao, C. T. Lim, Single molecule and multiple bond characterization of catch bond associated cytoadhesion in malaria. Sci Rep 7: 4208 (2017).
25. M. I. Love, W. Huber, S. Anders, Moderated estimation of fold change and dispersion for RNA-seq data with DESeq2. Genome Biology 15: 550 (2014).
26. A. M. Mabb, M. C. Judson, M. J. Zylka, B. D. Philpot, Angelman syndrome: insights into genomic imprinting and neurodevelopmental phenotypes. Trends Neurosci 34: 293-303 (2011).
27. J. L. Maciaszek, H. Soh, R. S. Walikonis, A. V. Tzingounis, G. Lykotrafitis, Topography of native SK channels revealed by force nanoscopy in living neurons. J Neurosci 32: 11435-11440 (2012).
28. McLeod, J. F., et al., GAL-021, a new intravenous BKCa-channel blocker, is well tolerated and stimulates ventilation in healthy volunteers. British J. Anaesthesia 113: 875-883 (2014).
29. J. R. Montgomery, A. L. Meredith, Genetic activation of BK currents in vivo generates bidirectional effects on neuronal excitability. Proc Natl Acad Sci USA 109: 18997-19002 (2012).
30. A. M. Pasca et al., Functional cortical neurons and astrocytes from human pluripotent stem cells in 3D culture. Nat Methods 12: 671-678 (2015).
31. K. Pelc, S. G. Boyd, G. Cheron, B. Dan, Epilepsy in Angelman syndrome. Seizure 17: 211-217 (2008).
32. J. Platkiewicz, R. Brette, A threshold equation for action potential initiation. PLoS Comput Biol 6: e1000850 (2010).
33. R. J. Racine, Modification of seizure activity by electrical stimulation. II. Motor seizure. Electroencephalogr Clin Neurophysiol 32: 281-294 (1972).
34. S. Ramamoorthy, Z. Nawaz, E6-associated protein (E6-AP) is a dual function coactivator of steroid hormone receptors. Nucl Recept Signal 6: e006 (2008).
35. J. Reimand et al., g:Profiler—a web server for functional interpretation of gene lists (2016 update). Nucleic Acids Research 44: W83-W89 (2016).
36. B. Sadikovic et al., Mutation Update for UBE3A variants in Angelman syndrome. Hum Mutat 35: 1407-1417 (2014).
37. N. E. Sanjana, O. Shalem, F. Zhang, Improved vectors and genome-wide libraries for CRISPR screening. Nat Methods 11: 783-784 (2014).
38. G. L. Sell, S. S. Margolis, From UBE3A to Angelman syndrome: a substrate perspective. Front Neurosci 9: 322 (2015).
39. A. Sergushichev, An algorithm for fast preranked gene set enrichment analysis using cumulative statistic calculation. bioRxiv, (2016).
40. M. S. Sidorov et al., Delta rhythmicity is a reliable EEG biomarker in Angelman syndrome: a parallel mouse and human analysis. J Neurodev Disord 9: 17 (2017).
41. J. F. Storm, Action potential repolarization and a fast after-hyperpolarization in rat hippocampal pyramidal cells. J Physiol 385: 733-759 (1987).
42. Strimmer, K. A unified approach to false discovery rate estimation. BMC Bioinformatics 9: 303 (2008).
43. A. Subramanian et al., Gene set enrichment analysis: A knowledge-based approach for interpreting genome-wide expression profiles. Proceedings of the National Academy of Sciences 102: 15545-15550 (2005).
44. J. Sun et al., UBE3A Regulates Synaptic Plasticity and Learning and Memory by Controlling SK2 Channel Endocytosis. Cell Rep 12: 449-461 (2015).
45. A. X. Sun et al., Direct Induction and Functional Maturation of Forebrain GABAergic Neurons from Human Pluripotent Stem Cells. Cell Rep 16: 1942-1953 (2016).
46. M. L. Wallace, A. C. Burette, R. J. Weinberg, B. D. Philpot, Maternal loss of Ube3a produces an excitatory/inhibitory imbalance through neuron type-specific synaptic defects. Neuron 74: 793-800 (2012).
46. M. L. Wallace, G. M. van Woerden, Y. Elgersma, S. L. Smith, B. D. Philpot, UBE3A loss increases excitability and blunts orientation tuning in the visual cortex of Angelman syndrome model mice. J Neurophysiol, 118(1): 634-646 (2017).
47. F. Yi et al., Autism-associated SHANK3 haploinsufficiency causes lh channelopathy in human neurons. Science 352(6286): aaf2669 (2016).
48. J. J. Yi et al., The autism-linked UBE3A T485A mutant E3 ubiquitin ligase activates the Wnt/beta-catenin pathway by inhibiting the proteasome. J Biol Chem 292: 12503-12515 (2017).
49. Y. Yu, Y. Shu, D. A. McCormick, Cortical action potential backpropagation explains spike threshold variability and rapid-onset kinetics. J Neurosci 28: 7260-7272 (2008).
50. Y. Zhang et al., Rapid single-step induction of functional neurons from human pluripotent stem cells. Neuron 78: 785-798 (2013).

1. A compound or composition comprising said compound for use in the prophylaxis or treatment of seizures caused by one or more UBE3A mutations in a subject, wherein the one or more UBE3A mutations cause a BK channelopathy and wherein the compound or composition is an antagonist of BK channel activity.

2.-3. (canceled)

4. The compound or composition of claim 1, wherein the subject has Angelman syndrome or a related autism spectrum disorder.

5. The compound or composition of claim 1, wherein the compound is selected from the group comprising Paxilline, IBTX, GAL021, small molecules, antisense molecules, aptamers, ribozymes, triplex forming molecules, small interfering RNA (siRNA), RNA interference (RNAi), and external guide sequences (EGS).

6. The composition of claim 1, comprising pharmaceutically acceptable salts or solvates, or pharmaceutically functional derivatives of said BK antagonist compound.

7. The composition of claim 1, comprising a BK channel antagonist compound with a pharmaceutically-acceptable adjuvant, diluent or carrier.

8. The composition of claim 1, wherein the composition is formulated for administration of a BK channel antagonist in the range of about 0.05 mg/kg to about 10 mg/kg, about 0.05 mg/kg to about 5 mg/kg, preferably about 0.3 mg/kg to about 3 mg/kg.

9.-13. (canceled)

14. A method of prophylaxis or treatment of seizures caused by one or more UBE3A mutations in a subject, comprising administering to a subject in need thereof a therapeutically effective amount of a compound or composition comprising an antagonist of BK channel activity.

15. The method of claim 14, wherein the one or more UBE3A mutations cause a BK channelopathy,

16. The method of claim 14, wherein the subject has Angelman syndrome or a related autism spectrum disorder.

17. The method of claim 14, wherein the antagonist of BK channel activity is selected from the group comprising Paxilline, IBTX, GAL021, small molecules, and functional nucleic acids such as antisense molecules, aptamers, ribozymes, triplex forming molecules, small interfering RNA (siRNA), RNA interference (RNAi), and external guide sequences (EGS).

18. The method of claim 14, wherein the composition comprises pharmaceutically acceptable salts or solvates, or pharmaceutically functional derivatives of said BK antagonist compound.

19. The method of claim 14, wherein the composition comprises a BK channel antagonist compound with a pharmaceutically-acceptable adjuvant, diluent or carrier.

20. The method of claim 14, wherein the therapeutically effective amount of the antagonist of BK channel activity is in the range of 0.05 mg/kg to about 10 mg/kg, about 0.05 mg/kg to about 5 mg/kg, preferably about 0.3 mg/kg to about 3 mg/kg.
